(19) United States
(12) Patent Application Publication
(10) Pub. No.: US 2023/0190755 A1
(43) Pub. Date: Jun. 22, 2023

(54) Topical formulations of PI3K-delta inhibitors

(71) Applicant: Incyte Corporation, Wilmington, DE (US)

(72) Inventors: Indushekhar Persaud, Wilmington, DE (US); Melissa Parker, Wilmington, DE (US); Paul Smith, Carlsbad, CA (US); Zheng Zhang, Irvine, CA (US)

(21) Appl. No.: 18/081,975

(22) Filed: Dec. 15, 2022

Related U.S. Application Data
(60) Provisional application No. 63/290,518, filed on Dec. 16, 2021.

(51) Int. Cl. A61K 31/519; A61K 9/06; A61K 9/00; A61K 47/10; A61K 47/36; A61K 47/18; A61P 17/06

(52) U.S. Cl. CPC A61K 31/519; A61K 9/06; A61K 9/0014; A61K 47/10; A61K 47/36; A61K 47/183; A61P 17/06

(57) ABSTRACT

The present application relates to pharmaceutical formulations for topical skin application comprising a PI3K-delta inhibitor, e.g., 4-(3-(-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and use in the treatment of skin disorders.

### TECHNICAL FIELD

The present disclosure is related to pharmaceutical formulations for topical skin application comprising a PI3K-delta inhibitor, e.g., 4-(3-(-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and use in the treatment of skin disorders.

### BACKGROUND

Phosphoinositide 3-kinase (PI3K) related pathways are important signalling pathways for many cellular functions such as growth control, metabolism, and translation initiation. Phosphoinositide 3-kinase inhibitors, PI3K inhibitors, function by inhibiting one or more of these pathways, such as by inhibiting phosphoinositide 3-kinase enzymes. Various PI3K inhibitors have been approved for the treatment of cancer, including idelalisib, copanlisib, and duvelisib.

Given the usefulness of PI3K inhibitors there is a need for improved topical formulations of PI3K inhibitors. In particular, there is a need for stable, easily applied formulations for PI3K inhibitors, e.g., PI3K delta inhibitors, with good skin permeation characteristics. The formulations of the present invention, as well the methods described herein, are directed toward this need and other ends.

### SUMMARY

The present application provides, inter alia, pharmaceutical compositions, suitable for topical skin application to a human patient with a skin disorder, comprising:

(1) a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof; and

(2) a means for effecting skin permeation of the therapeutic agent or pharmaceutically acceptable salt thereof to the patient.

The present application further provides, inter alia, pharmaceutical compositions, comprising a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof.

The present application further provides methods of treating a skin disorder in a patient in need thereof, comprising applying a pharmaceutical composition described herein, e.g., an oil in water emulsion, to an area of skin of the patient.

The present application further provides a method of treating a skin disorder in a human patient in need thereof, comprising applying to the patient's skin a pharmaceutically acceptable composition comprising a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and a means of effecting skin permeation of the therapeutic agent to the patient, wherein the treating step is one or more of (i) inhibiting the skin disorder, and (b) ameliorating the skin disorder.

The present disclosure further provides a pharmaceutical composition described herein, for use in any of the methods described herein.

The present disclosure further provides use of a pharmaceutical composition described herein, for the preparation of a medicament for use in any of the methods described herein.

### DESCRIPTION OF DRAWINGS

FIG. 1 depicts ear thickness in a chronic CXCL13 induced B cell mouse model upon dosing with topical formulations of varying concentrations of Compound A as described in Example 13.

FIG. 2 depicts ear thickness in a chronic healthy mouse model upon dosing with topical formulations of Compound A with varying amounts of PEG200 as described in Example 14.

FIG. 3 depicts ear redness in a chronic healthy mouse model upon dosing with topical formulations of Compound A with varying amounts of PEG200 as described in Example 14.

FIG. 4 depicts a custom redness scale created for the redness observations as described in Example 14. The beginning of the scale (1) is roughly equivalent to a healthy, untreated Balb/C mouse ear and increases in redness with increasing values. Redness scale 5 represents an exceptionally red Balb/C mouse ear.

FIG. 5 depicts CD4+ T cell count of auricular lymph node by flow cytometry in a chronic healthy mouse model upon dosing with topical formulations of Compound A with varying amounts of PEG200 as described in Example 14.

FIG. 6 depicts CD45+ lymphocyte frequency of auricular lymph node by flow cytometry in a chronic healthy mouse model upon dosing with topical formulations of Compound A with varying amounts of PEG200 as described in Example 14.

FIG. 7 depicts ear thickness in an acute FITC induced dermatitis mouse model upon dosing with topical formulations of Compound A as described in Example 18.

FIG. 8 depicts ear thickness in a chronic FITC induced dermatitis mouse model upon dosing with topical formulations of Compound A as described in Example 18.

FIG. 9 depicts the whole blood frequency of CD19+ B cells upon dosing with topical formulations of Compound A as described in Example 18.

FIG. 10 depicts the whole blood frequency of CD3+ T cells upon dosing with topical formulations of Compound A as described in Example 18.

FIG. 11 depicts ear thickness changes from untreated ear in healthy mice upon dosing with topical formulations to investigate the inflammatory response to various levels of propylene glycol for topical formulation with and without PEG400 as described in Example 14.

FIG. 12 depicts CD4+ T cell count of auricular lymph node by flow cytometry in a healthy mice upon dosing with topical formulations of Compound A with and without xanthan gum (XG) as described in Example 21.

### DETAILED DESCRIPTION

The present disclosure describes a pharmaceutical composition comprising 4-(3-(-1-(4-amino-3-methyl-1H-pyrazolo[3,4-d]pyrimidin-1-yl)ethyl)-5-chloro-2-ethoxy-6-fluorophenyl)pyrrolidin-2-one, and more particularly (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one (i.e., Compound A), or a pharmaceutically acceptable salt thereof, which is suitable for topical administration and treatment of skin disorders.

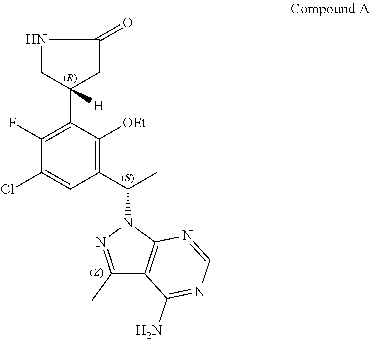

A selective PI3Kδ inhibitor, (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, and pharmaceutically acceptable salts thereof, have previously been described in U.S. Pat. Nos. 9,199,982, 9,707,233, 9,730,939, 9,932,341, 10,064,866, 10,092,570, 10,336,759, 10,376,513, 10,646,492, 10,675,284, and U.S. Application Publication Nos.: 20190308979, 20190365764, 20200323858, and 20210093638, the disclosure of each of which is incorporated herein by reference in its entirety.

The present disclosure further describes a pharmaceutical composition, suitable for topical skin application to a human patient with a skin disorder, comprising:
(1) a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and
(2) a means for effecting skin permeation of the therapeutic agent or pharmaceutically acceptable salt thereof to the patient.

In some embodiments, the composition is in the form of an emulsion, a cream, an ointment, a film forming foam, a gel, a suppository, a lotion, a transdermal patch, or a hydrogel. In some embodiments, the composition is suitable for topical administration to a human patient.

In some embodiments, the composition is an oil-in-water emulsion.

In some embodiments, the oil-in-water emulsion is a cream.

In some embodiments, the oil-in-water emulsion comprises water, an oil component, and an emulsifier component.

In some embodiments, the oil component comprises about 10% to about 40% by weight of the composition.

In some embodiments, the oil component comprises about 15% to about 25% by weight of the composition. In some embodiments, the oil component comprises about 21% to about 23% by weight of the composition. In some embodiments, the oil component comprises about 22% by weight of the composition.

In some embodiments, the oil component comprises one or more substances independently selected from petrolatums, fatty alcohols, mineral oils, triglycerides, and silicone oils. In some embodiments, the oil component comprises one or more substances independently selected from white petrolatum, cetyl alcohol, stearyl alcohol, light mineral oil, and medium chain triglycerides.

In some embodiments, the oil component comprises an occlusive agent component. In some embodiments, the occlusive agent component is present in an amount of about 1% to about 10% by weight of the composition. In some embodiments, the occlusive agent component is present in an amount of about 5% to about 10% by weight of the composition. In some embodiments, the occlusive agent component is present in an amount of about 6% to about 8% by weight of the composition. In some embodiments, the occlusive agent component is present in an amount of about 7% by weight of the composition.

In some embodiments, the occlusive agent component comprises a petrolatum. In some embodiments, the occlusive agent component comprises white petrolatum.

In some embodiments, the oil component comprises a stiffening agent component.

In some embodiments, the stiffening agent component is present in an amount of about 1% to about 8% by weight of the composition. In some embodiments, the stiffening agent component is present in an amount of about 4% to about 6% by weight of the composition. In some embodiments, the stiffening agent component is present in an amount of about 4% to about 5% by weight of the composition. In some embodiments, the stiffening agent component is present in an amount of about 5% by weight of the composition.

In some embodiments, the stiffening agent component comprises one or more substances independently selected from fatty alcohols. In some embodiments, the stiffening agent component comprises one or more substances independently selected from C₁₂₋₂₀ fatty alcohols. In some embodiments, the stiffening agent component comprises one or more substances independently selected from C₁₆₋₁₈ fatty alcohols. In some embodiments, the stiffening agent component comprises one or more substances independently selected from cetyl alcohol and stearyl alcohol. In some embodiments, the stiffening agent component comprises cetyl alcohol and stearyl alcohol.

In some embodiments, the oil component comprises an emollient component.

In some embodiments, the emollient component is present in an amount of about 5% to about 15% by weight of the composition.

In some embodiments, the emollient component is present in an amount of about 8% to about 12% by weight of the composition.

In some embodiments, the emollient component is present in an amount of about 9% to about 11% by weight of the composition.

In some embodiments, the emollient component is present in an amount of about 10% by weight of the composition.

In some embodiments, the emollient component comprises one or more substances independently selected from mineral oils and triglycerides. In some embodiments, the emollient component comprises one or more substances independently selected from light mineral oil and medium chain triglycerides. In some embodiments, the emollient component comprises light mineral oil and medium chain triglycerides.

In some embodiments, the water is present in an amount of about 30% to about 70% by weight of the composition.

In some embodiments, the water is present in an amount of about 40% to about 60% by weight of the composition.

In some embodiments, the water is present in an amount of about 50% to about 60% by weight of the composition.

In some embodiments, the water is present in an amount of about 55% to about 57% by weight of the composition.

In some embodiments, the water is present in an amount of about 56% by weight of the composition.

In some embodiments, the emulsifier component is present in an amount of about 1% to about 10% by weight of the composition.

In some embodiments, the emulsifier component is present in an amount of about 2% to about 6% by weight of the composition.

In some embodiments, the emulsifier component is present in an amount of about 3% to about 5% by weight of the composition.

In some embodiments, the emulsifier component is present in an amount of about 4% by weight of the composition.

In some embodiments, the pharmaceutical composition comprises an emulsifier component and a stiffening agent component, wherein the combined amount of emulsifier component and stiffening agent component is at least about 9% by weight of the composition.

In some embodiments, the emulsifier component comprises one or more substances independently selected from glyceryl fatty esters and sorbitan fatty esters. In some embodiments, the emulsifier component comprises one or more substances independently selected from glyceryl stearate and polysorbate 20. In some embodiments, the emulsifier component comprises one or more substances independently selected from glyceryl monostearate, glyceryl distearate, and polysorbate 20. In some embodiments, the emulsifier component comprises glyceryl monostearate, glyceryl distearate, and polysorbate 20.

In some embodiments, the pharmaceutical composition further comprises a stabilizing agent component. In some embodiments, the stabilizing agent component is present in an amount of about 0.01% to about 2% by weight of the composition. In some embodiments, the stabilizing agent component is present in an amount of about 0.05% to about 1% by weight of the composition. In some embodiments, the stabilizing agent component is present in an amount of about 0.1% to about 0.5% by weight of the composition. In some embodiments, the stabilizing agent component is present in an amount of about 0.2% to about 0.4% by weight of the composition. In some embodiments, the stabilizing agent component is present in an amount of about 0.35% by weight of the composition. In some embodiments, the stabilizing agent component is present in an amount of about 0.4% by weight of the composition.

In some embodiments, the stabilizing agent component comprises one or more independently selected polysaccharides. In some embodiments, the stabilizing agent component comprises xanthan gum.

In some embodiments, the pharmaceutical composition further comprises a solvent component. In some embodiments, the solvent component is present in an amount of about 10% to about 30% by weight of the composition. In some embodiments, the solvent component is present in an amount of about 15% to about 20% by weight of the composition. In some embodiments, the solvent component is present in an amount of about 16% to about 18% by weight of the composition. In some embodiments, the solvent component is present in an amount of about 17% by weight of the composition. In some embodiments, the solvent component is present in an amount of about 6% to about 20% by weight of the composition. In some embodiments, the solvent component is present in an amount of about 5% by weight of the composition. In some embodiments, the solvent component is present in an amount of about 15% by weight of the composition.

In some embodiments, the solvent component comprises one or more substances independently selected from alkylene glycols and polyalkylene glycols. In some embodiments, the solvent component comprises one or more substances independently selected from propylene glycol and polyethylene glycol. In some embodiments, the solvent component comprises propylene glycol and polyethylene glycol. In some embodiments, the polyethylene glycol is PEG200. In some embodiments, the polyethylene glycol is PEG300 or PEG400. In some embodiments, the polyethylene glycol is PEG300. In some embodiments, the polyethylene glycol is PEG400.

In some embodiments, the propylene glycol is present in an amount of about 5% to about 15% by weight of the composition. In some embodiments, the propylene glycol is present in an amount of about 5% by weight of the composition. In some embodiments, the propylene glycol is present in an amount of about 10% by weight of the composition. In some embodiments, the propylene glycol is present in an amount of about 15% by weight of the composition. In some embodiments, the polyethylene glycol is present in an amount of about 1% to about 7% by weight of the composition. In some embodiments, the polyethylene glycol is present in an amount of about 1% to about 5% by weight of the composition. In some embodiments, the polyethylene glycol is present in an amount of about 1% by weight of the composition. In some embodiments, the polyethylene glycol is present in an amount of about 5% by weight of the composition. In some embodiments, the polyethylene glycol is present in an amount of about 7% by weight of the composition.

In some embodiments, the therapeutic agent is present in an amount of about 0.001% to about 2.0% by weight of the composition on a free base basis. In some embodiments, the therapeutic agent is present in an amount of about 0.001% to about 1.0% by weight of the composition on a free base basis. In some embodiments, the therapeutic agent is present in an amount of about 0.005% to about 0.05% by weight of the composition on a free base basis. In some embodiments, the therapeutic agent is present in an amount of about 0.005% to about 0.02% by weight of the composition on a free base basis.

In some embodiments, the pharmaceutical composition comprises:

about 30% to about 70% of water by weight of the composition;

about 10% to about 40% of an oil component by weight of the composition;

about 1% to about 10% of an emulsifier component by weight of the composition;

about 10% to about 30% of a solvent component by weight of the composition;

about 0.01% to about 2% of a stabilizing agent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 40% to about 60% of water by weight of the composition;

about 15% to about 25% of an oil component by weight of the composition;

about 2% to about 6% of an emulsifier component by weight of the composition;

about 15% to about 20% of a solvent component by weight of the composition;

about 0.05% to about 1% of a stabilizing agent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 50% to about 60% of water by weight of the composition;

about 21% to about 23% of an oil component by weight of the composition;

about 3% to about 5% of an emulsifier component by weight of the composition;

about 16% to about 18% of a solvent component by weight of the composition;

about 0.1% to about 0.5% of a stabilizing agent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 56% of water by weight of the composition;

about 22% of an oil component by weight of the composition;

about 4% of an emulsifier component by weight of the composition;

about 17% of a solvent component by weight of the composition;

about 0.35% of a stabilizing agent component by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the oil component comprises one or more substances independently selected from petrolatums, fatty alcohols, mineral oils, triglycerides, and silicone oils; the emulsifier component comprises one or more substances independently selected from glyceryl fatty esters and sorbitan fatty esters; the solvent component comprises one or more substances independently selected from alkylene glycols and polyalkylene glycols; and the stabilizing agent component comprises one or more independently selected polysaccharides.

In some embodiments, the oil component comprises one or more substances independently selected from white petrolatum, cetyl alcohol, stearyl alcohol, light mineral oil, and medium chain triglycerides; the emulsifier component comprises one or more substances independently selected from glyceryl stearate and polysorbate 20; the solvent component comprises one or more substances independently selected from propylene glycol and polyethylene glycol; and the stabilizing agent component comprises xanthan gum.

In some embodiments, the pharmaceutical composition comprises:

about 30% to about 70% of water by weight of the composition;

about 1% to about 10% of an occlusive agent component by weight of the composition;

about 1% to about 8% of a stiffening agent component by weight of the composition;

about 5% to about 15% of an emollient component by weight of the composition;

about 1% to about 10% of an emulsifier component by weight of the composition;

about 0.01% to about 2% of a stabilizing agent component by weight of the composition;

about 10% to about 30% of a solvent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 40% to about 60% of water by weight of the composition;

about 5% to about 10% of an occlusive agent component by weight of the composition;

about 4% to about 6% of a stiffening agent component by weight of the composition;

about 8% to about 12% of an emollient component by weight of the composition;

about 2% to about 6% of an emulsifier component by weight of the composition;

about 0.05% to about 1% of a stabilizing agent component by weight of the composition;

about 15% to about 20% of a solvent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 50% to about 60% of water by weight of the composition;

about 6% to about 8% of an occlusive agent component by weight of the composition;

about 4% to about 5% of a stiffening agent component by weight of the composition;

about 9% to about 11% of an emollient component by weight of the composition;

about 3% to about 5% of an emulsifier component by weight of the composition;

about 0.1% to about 0.5% of a stabilizing agent component by weight of the composition;

about 16% to about 18% of a solvent component by weight of the composition; and

about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises:

about 56% of water by weight of the composition;

about 7% of an occlusive agent component by weight of the composition;

about 5% of a stiffening agent component by weight of the composition;

about 10% of an emollient component by weight of the composition;

about 4% of an emulsifier component by weight of the composition;

about 0.35% of a stabilizing agent component by weight of the composition;

about 17% of a solvent component by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

In some embodiments, the pharmaceutical composition comprises an emulsifier component and a stiffening agent component, wherein the combined amount of emulsifier component and stiffening agent component is at least about 9% by weight of the composition.

In some embodiments:

the occlusive agent component comprises a petrolatum;

the stiffening agent component comprises one or more substances independently selected from one or more fatty alcohols;

the emollient component comprises one or more substances independently selected from mineral oils and triglycerides;

the emulsifier component comprises one or more substances independently selected from glyceryl fatty esters and sorbitan fatty esters;

the stabilizing agent component comprises one or more substances independently selected from polysaccharides; and

the solvent component comprises one or more substances independently selected from alkylene glycols and polyalkylene glycols.

In some embodiments:

the occlusive agent component comprises white petrolatum;

the stiffening agent component comprises one or more substances independently selected from cetyl alcohol and stearyl alcohol;

the emollient component comprises one or more substances independently selected from light mineral oil and medium chain triglycerides;

the emulsifier component comprises one or more substances independently selected from glyceryl stearate and polysorbate 20;

the stabilizing agent component comprises xanthan gum; and

the solvent component comprises one or more substances independently selected from propylene glycol and polyethylene glycol.

In some embodiments:

the occlusive agent component comprises white petrolatum;

the stiffening agent component comprises cetyl alcohol and stearyl alcohol;

the emollient component comprises light mineral oil and medium chain triglycerides;

the emulsifier component comprises glyceryl stearate and polysorbate 20;

the stabilizing agent component comprises xanthan gum; and

the solvent component comprises propylene glycol and polyethylene glycol.

In some embodiments, the therapeutic agent is present in an amount of about 0.001% by weight of the composition on a free base basis. In some embodiments, the therapeutic agent is present in an amount of about 0.01% by weight of the composition on a free base basis. In some embodiments, the therapeutic agent is present in an amount of about 0.1% by weight of the composition on a free base basis.

In some embodiments, the therapeutic agent is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt.

In some embodiments, the pharmaceutical composition further comprises an antimicrobial preservative component. In some embodiments, the antimicrobial preservative component is present in an amount of about 0.05% to about 2% by weight of the composition. In some embodiments, the antimicrobial preservative component is present in an amount of about 0.1% to about 1% by weight of the composition. In some embodiments, the antimicrobial preservative component is present in an amount of about 0.4% to about 0.6% by weight of the composition. In some embodiments, the antimicrobial preservative component is present in an amount of about 0.5% by weight of the composition.

In some embodiments, the antimicrobial preservative component comprises phenoxyethanol.

In some embodiments, the pharmaceutical composition further comprises a chelating agent component. In some embodiments, the chelating agent component is present in an amount of about 0.01% to about 0.1% by weight of the composition. In some embodiments, the chelating agent component is present in an amount of about 0.04% to about 0.06% by weight of the composition. In some embodiments, the chelating agent component is present in an amount of about 0.05% by weight of the composition.

In some embodiments, the chelating agent component comprises disodium EDTA.

In some embodiments, the pharmaceutical composition comprises:

about 5% to about 10% of white petrolatum by weight of the composition;

about 1% to about 5% of cetyl alcohol by weight of the composition;

about 1% to about 5% of stearyl alcohol by weight of the composition;

about 1% to about 5% of light mineral oil by weight of the composition;

about 4% to about 8% of medium chain triglycerides by weight of the composition;

about 1% to about 5% of glyceryl mono and distearate by weight of the composition;

about 0.1% to about 2% of polysorbate 20 by weight of the composition;

about 0.1% to about 2% of xanthan gum by weight of the composition;

about 5% to about 10% of PEG300 by weight of the composition;

about 5% to about 15% of propylene glycol by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 7% of white petrolatum by weight of the composition;

about 3% of cetyl alcohol by weight of the composition;

about 2% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1% of polysorbate 20 by weight of the composition;

about 0.35% of xanthan gum by weight of the composition;

about 7% of PEG300 by weight of the composition;

about 10% of propylene glycol by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 7% of white petrolatum by weight of the composition;

about 3% of cetyl alcohol by weight of the composition;

about 1.75% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1.25% of polysorbate 20 by weight of the composition;

about 0.35% of xanthan gum by weight of the composition;

about 7% of PEG300 by weight of the composition;

about 10% of propylene glycol by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 56% to about 57% of water by weight of the composition;

about 7% of white petrolatum by weight of the composition;

about 3% of cetyl alcohol by weight of the composition;

about 1.75% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1.25% of polysorbate 20 by weight of the composition;

about 0.35% of xanthan gum by weight of the composition;

about 7% of PEG300 by weight of the composition;

about 10% of propylene glycol by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 56% of water by weight of the composition;

about 7% of white petrolatum by weight of the composition;

about 3% of cetyl alcohol by weight of the composition;

about 2% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1% of polysorbate 20 by weight of the composition;

about 0.35% of xanthan gum by weight of the composition;

about 7% of PEG300 by weight of the composition;

about 10% of propylene glycol by weight of the composition; and

about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 56% of water by weight of the composition;

about 7% of white petrolatum by weight of the composition;

about 3% of cetyl alcohol by weight of the composition;

about 2% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1% of polysorbate 20 by weight of the composition;

about 0.35% of xanthan gum by weight of the composition;

about 7% of PEG300 by weight of the composition;

about 10% of propylene glycol by weight of the composition;

about 0.05% of disodium EDTA by weight of the composition;

about 0.5% of phenoxyethanol by weight of the composition; and about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition comprises:

about 56% of water by weight of the composition;

about 7% of white petrolatum by weight of the composition;

about 3% of cetyl alcohol by weight of the composition;

about 2% of stearyl alcohol by weight of the composition;

about 4% of light mineral oil by weight of the composition;

about 6% of medium chain triglycerides by weigh of the composition;

about 3% of glyceryl mono and distearate by weight of the composition;

about 1% of polysorbate 20 by weight of the composition;

about 0.35% of xanthan gum by weight of the composition;

about 7% of PEG300 by weight of the composition;

about 10% of propylene glycol by weight of the composition;

about 0.05% of disodium EDTA by weight of the composition;

about 0.5% of phenoxyethanol by weight of the composition; and

about 0.1% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

In some embodiments, the pharmaceutical composition is a cream, gel, hydrogel, aerosolized foam, non-aerosolized foam, film forming spray, or ointment. In some embodiments, the pharmaceutical composition is a cream.

In some embodiments, the skin disorder is an immune mediated dermatological disease.

In some embodiments, the immune mediated dermatological disease is cutaneous T cell lymphoma, atopic dermatitis, psoriasis, contact dermatitis, chronic hand eczema, hidradenitis suppurativa, lichen planus, acne, skin blistering disease, chronic urticaria, or cold induced urticaria.

In some embodiments, the cutaneous T cell lymphoma is mycosis fungoides.

In some embodiments, the immune mediated dermatological disease is mycosis fungoides, atopic dermatitis, psoriasis, contact dermatitis, chronic hand eczema, hidradenitis suppurativa, lichen planus, acne, skin blistering disease, chronic urticaria, or cold induced urticaria.

In some embodiments, the immune mediated dermatological disease is atopic dermatitis, psoriasis, contact dermatitis, chronic hand eczema, hidradenitis suppurativa, lichen planus, acne, skin blistering disease, chronic urticaria, or cold induced urticaria.

In some embodiments, the immune mediated dermatological disease is cutaneous T cell lymphoma.

In some embodiments, the immune mediated dermatological disease is mycosis fungoides.

In some embodiments, the immune mediated dermatological disease is atopic dermatitis.

In some embodiments, the immune mediated dermatological disease is psoriasis.

In some embodiments, the oil-in-water emulsion is a cream. In some embodiments, the oil-in-water emulsion is a gel, hydrogel, aerosolized foam, non-aerosolized foam, film forming spray, or ointment. In one embodiment, the topical formulations of the present disclosure are an oil-in-water emulsion topical cream containing 0.01% w/w of Compound A. In one embodiment, the topical formulations of the present disclosure are an oil-in-water emulsion topical cream containing 0.1% w/w of Compound A. The formulations may also contain purified water, propylene glycol, polyethylene glycol, white petroleum, medium chain triglycerides, light mineral oil, glyceryl mono and distearate, cetyl alcohol, stearyl alcohol, polysorbate, phenoxyethanol, xanthan gum, and disodium ethylenediaminetetraacetic acid.

The formulations described herein were found to have good skin permeation in human skin ex-vivo models.

The formulations described herein were found to have good stability over a 12 week period when stored at 25° C./60% RH.

In transport studies with human skin, the formulations described herein also displayed a trend of increased permeability when the strength of the formulation was increased from 0.01% w/w to 0.1% w/w.

Further, the formulations described herein are relatively simple to manufacture with a repeatable process of formulation. The resultant product is easily packaged. The formulations appear to have good stability and relatively consistent permeation profiles.

In some embodiments, the non-PI3K inhibitor excipients of the formulation do not contribute to an inflammatory response. In some embodiments, the non-PI3K inhibitor excipients of the formulation minimally contribute to an inflammatory response.

As used herein, the term “emulsifier component” refers, in one aspect, to a substance, or mixtures of substances, that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the term “occlusive agent component” refers to a hydrophobic agent, or mixtures of hydrophobic agents, that form an occlusive film on skin that reduces transepidermal water loss (TEWL) by preventing evaporation of water from the stratum corneum.

As used herein, the term “stiffening agent component” refers to a substance, or mixture of substances, that increases the viscosity and/or consistency of the formulation or improves the rheology of the formulation.

As used herein, the term “emollient component” refers to an agent, or a mixture of agents, that softens or soothes the skin or soothes an irritated internal surface.

As used herein, the term “stabilizing agent component” refers to a substance, or mixture of substances, that improves the stability of the pharmaceutical formulation and/or the compatibility of the components in the formulation. In some embodiments, the stabilizing agent component prevents agglomeration of the emulsion and stabilizes the droplets in the oil-in-water emulsion.

As used herein, the term “solvent component” is a liquid substance, or mixture of liquid substances, capable of dissolving the PI3K-delta inhibitor, in particular (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, or other substances in the formulation.

As used herein, the phrase “antimicrobial preservative component” is a substance, or mixtures of substances, which inhibits microbial growth in the formulation.

As used herein, the phrase “chelating agent component” refers to a compound, or mixtures of compounds, that has the ability to bind strongly with metal ions.

As used herein, the phrase “skin permeation” refers to the transport of a substance through the skin.

As used herein, the phrase “effecting skin permeation of the therapeutic agent” refers to causing the therapeutic agent to transport through the skin.

As used herein, in the phrase “means for effecting skin permeation of the therapeutic agent”, the “means” refers to all known topical formulations, e.g., transdermal patches, ointments, lotions, creams, gels, hydrogels, drops, suppositories, sprays, film forming sprays, liquids, powders, aerosolized foams, non-aerosolized foams, as well as topical formulations described herein.

As used herein, the term “skin” refers to one or more layers or sublayers of skin, including but not limited to the epidermis (including but not limited to basal cell layer (also known as stratum germinativum), squamous cell layer (also known as stratum spinosum or “spiny layer”), stratum granulosum, stratum lucidum, and stratum corneum sublayers), the dermis (including but not limited to papillary and reticular sublayers), and the subcutis (also known as the hypodermis or subcutaneous layer). In some embodiments, the skin comprises the epidermis. In some embodiments, the skin comprises the epidermis and the dermis. In some embodiments, the skin comprises the epidermis, the dermis and the subcutis. In some embodiments, the skin comprises the dermis. In some embodiments, the skin comprises the subcutis.

In some embodiments, the transport of the therapeutic agent through the skin is indicated by the presence of the therapeutic agent in the skin, i.e., one or more layers or sublayers of skin, after application of a topical formulation comprising the therapeutic agent to the surface of the skin. In some embodiments, the therapeutic agent is present in the skin in an amount that is about 0.01% to about 15% of the dose of the therapeutic agent applied to the surface of the skin. In some embodiments, the therapeutic agent is present in the skin in an amount that is about 0.01% to about 10% of the dose of the therapeutic agent applied to the surface of the skin. In some embodiments, the therapeutic agent is present in the skin in an amount that is about 0.01% to about 5% of the dose of the therapeutic agent applied to the surface of the skin. In some embodiments, the therapeutic agent is present in the skin in an amount that is about 0.1% to about 10% of the dose of the therapeutic agent applied to the surface of the skin. In some embodiments, the therapeutic agent is present in the skin in an amount that is about 0.1% to about 5% of the dose of the therapeutic agent applied to the surface of the skin. In some embodiments, the therapeutic agent is present in the skin in an amount that is about 1% to about 10% of the dose of the therapeutic agent applied to the surface of the skin. In some embodiments, the therapeutic agent is present in the skin in an amount that is about 1% to about 5% of the dose of the therapeutic agent applied to the surface of the skin. In some embodiments, the therapeutic agent is present in the skin in an amount that is about 0.5% to about 3% of the dose of the therapeutic agent applied to the surface of the skin. In some embodiments, the therapeutic agent is present in the skin in an amount that is about 0.5% to about 2% of the dose of the therapeutic agent applied to the surface of the skin. In some embodiments, the therapeutic agent is present in the skin in an amount that is about 0.8% of the dose of the therapeutic agent applied to the surface of the skin. In some embodiments, the therapeutic agent is present in the skin in an amount that is about 2% of the dose of the therapeutic agent applied to the surface of the skin.

In some embodiments, the transport of the therapeutic agent is indicated by the amount present in the skin, i.e., one or more layers or sublayers of skin, after a period of time following application of a topical formulation comprising the therapeutic agent to the surface of the skin. In some embodiments, the period of time is from about 5 minutes to about 24 hours. In some embodiments, the period of time is from about 1 hour to about 24 hours. In some embodiments, the period of time is from about 6 hours to about 24 hours. In some embodiments, the period of time is about 1 hour. In some embodiments, the period of time is about 2 hours. In some embodiments, the period of time is about 4 hours. In some embodiments, the period of time is about 6 hours. In some embodiments, the period of time is about 8 hours. In some embodiments, the period of time is about 10 hours. In some embodiments, the period of time is about 12 hours. In some embodiments, the period of time is about 14 hours. In some embodiments, the period of time is about 16 hours. In some embodiments, the period of time is about 18 hours. In some embodiments, the period of time is about 20 hours. In some embodiments, the period of time is about 22 hours. In some embodiments, the period of time is about 24 hours.

In some embodiments, the transport of the therapeutic agent is indicated by the speed of penetration of the therapeutic agent into the skin, i.e., one or more layers or sublayers of skin, after application of a topical formulation comprising the therapeutic agent to the surface of the skin. In some embodiments, the speed of penetration is measured by skin flux.

As used herein, “skin flux” is the rate of flow of the therapeutic agent through the skin, i.e., one or more layers or sublayers of skin, upon application of the topical formulation comprising the therapeutic agent. Skin flux is often measured in terms of ng/cm²/h. In some embodiments the skin flux of the therapeutic agent is about 0.01 ng/cm²/h to about 1000 ng/cm²/h. In some embodiments the skin flux of the therapeutic agent is about 0.1 ng/cm²/h to about 1000 ng/cm²/h. In some embodiments the skin flux of the therapeutic agent is about 0.1 ng/cm²/h to about 100 ng/cm²/h. In some embodiments the skin flux of the therapeutic agent is about 0.1 ng/cm²/h to about 10 ng/cm²/h. In some embodiments the skin flux of the therapeutic agent is about 1 ng/cm²/h to about 10 ng/cm²/h. In some embodiments the skin flux of the therapeutic agent is about 1 ng/cm²/h to about 5 ng/cm²/h. In some embodiments the skin flux of the therapeutic agent is about 1 ng/cm²/h. In some embodiments the skin flux of the therapeutic agent is about 5 ng/cm²/h.

Measurement of the transport parameters can be performed by standard skin permeation experiments known in the art.

The compounds of the present disclosure also include pharmaceutically acceptable salts of the compounds disclosed herein. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

It will also be understood that compounds described herein may exist in solvated forms, for example hydrated, as well as unsolvated forms. It will further be understood that the present invention encompasses all such solvated forms of the compounds.

As used herein, the phrases “topical formulations” and “pharmaceutical compositions suitable for topical skin application” are interchangeable.

As used herein, “% by weight of the formulation” means the percent concentration of the component in the formulation is on weight/weight basis. For example, 1% w/w of component A=[(mass of component A)/(total mass of the formulation)]×100.

As used herein, “% by weight of the formulation on a free base basis” of the PI3K-delta inhibitor, in particular (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or pharmaceutically acceptable salt thereof” means that the % w/w is calculated based on the weight of the PI3K-delta inhibitor, in particular (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one in the total formulation.

In some embodiments, the components are present in the ranges specified (e.g., the term “about” is not present). In some embodiments, “about” refers to +10% of the value.

As will be appreciated, some components of the pharmaceutical formulations described herein can possess multiple functions. For example, a given substance may act as both an emulsifying agent component and a stabilizing agent. In some such cases, the function of a given component can be considered singular, even though its properties may allow multiple functionality. In some embodiments, each component of the formulation comprises a different substance or mixture of substances.

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobehenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula —O— alkylene-. In some embodiments, the polyalkylene glycol is polyethylene glycol.

As used herein, the term, “sorbitan fatty ester” includes products derived from sorbitan or sorbitol and fatty acids and, optionally, poly(ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE(20) sorbitan monolaurate), 21 (POE(4) sorbitan monolaurate), 40 (POE(20) sorbitan monopalmitate), 60 (POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

In some embodiments, the glyceryl stearate comprises glyceryl monostearate and glyceryl distearate.

As used herein, the term “triglycerides” refers to a triglyceride of a fatty acid. In some embodiments, the triglyceride is medium chain triglycerides.

As used herein, the term “alkylene glycol” refers to a group of formula —O— alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

As used herein, the term “qs” refers to a quantity sufficient to make.

### Methods of Use

The pharmaceutical compositions of the present disclosure are useful in treating skin disorders, e.g., via topical administration to an area of skin of a patient in need thereof.

In some embodiments, the pharmaceutical compositions of the present disclosure are useful in treating, preventing or reducing atopic dermatitis and other immune mediated dermatological diseases (e.g., psoriasis, hidradenitis suppurativa, vitiligo, alopecia areata, contact dermatitis, chronic hand eczema, lichen planus, acne, skin blistering disease, chronic urticaria, and cold induced urticaria).

The present disclosure further describes a method of treating a skin disorder in a patient in need thereof, comprising applying a pharmaceutical composition described herein to an area of skin of the patient.

The present disclosure further describes a method of treating a skin disorder in a human patient in need thereof, comprising applying to the patient's skin a pharmaceutically acceptable composition comprising a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and a means of effecting skin permeation of the therapeutic agent to the patient, wherein the treating step is one or more of (i) inhibiting the skin disorder, and (b) ameliorating the skin disorder.

In some embodiments, the skin disorder is an immune mediated dermatological disease. In some embodiments, the immune mediated dermatological disease is atopic dermatitis, psoriasis, contact dermatitis, chronic hand eczema, hidradenitis suppurativa, lichen planus, acne, skin blistering disease, chronic urticaria, or cold induced urticaria. In some embodiments, the immune mediated dermatological disease is atopic dermatitis. In some embodiments, the immune mediated dermatological disease is psoriasis.

In one embodiment, the topical formulations of the present disclosure can be used to treat a patient with atopic dermatitis. Atopic dermatitis is a chronic, inflammatory dermatological disease affecting 10% to 20% of children and up to 10% of the adult population per year. Atopic dermatitis is a heterogeneous disease involving environmental factors and genetic susceptibilities. The PI3Kδ pathway is important for immune cell function, including regulation of development, activation and differentiation of both B cells and T cells. Patients with AD have been found to have aberrantly activated PI3K/AKT pathway in peripheral T cells.

In one embodiment, the topical formulations of the present disclosure can be used to treat a patient with psoriasis. Psoriasis is a chronic inflammatory disease affecting approximately 2% to 3% of the population. It is often described in general terms as epidermal hyperproliferation induced by dermal infiltration of activated lymphocytes and the local release of various growth factors and cytokines. Psoriasis is a disorder of both the innate and adaptive immune arms of the immune system, involving T cells, dendritic cells, and keratinocytes as important players. The centrality of Th17 cells and an enhanced ILHard hyphen17 response have been confirmed with the use of ILHard hyphen17 pathway-targeting therapeutics.

The present disclosure further provides a pharmaceutical composition described herein, for use in any of the methods described herein.

The present disclosure further provides use of a pharmaceutical composition described herein, for the preparation of a medicament for use in any of the methods described herein.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the phrase “inhibiting the skin disorder” refers to arresting further development of the pathology and/or symptomatology of a skin disorder described herein.

As used herein, the phrase “ameliorating the skin disorder” refers to reversing the pathology and/or symptomatology of a skin disorder described herein.

In some embodiments, the pharmaceutical compositions of the present disclosure are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents such as, for example, chemotherapeutics, anti-inflammatory agents, steroids, immunosuppressants, as well as Bcr-Abl, Flt-3, EGFR, HER2, JAK (e.g., JAK1 or JAK2), c-MET, VEGFR, PDGFR, cKit, IGF-1R, RAF, FAK, Akt, mTOR, PIM, and AKT (e.g., AKT1, AKT2, or AKT3) kinase inhibitors such as, for example, those described in WO 2006/056399, or other agents such as, therapeutic antibodies can be used in combination with the compounds of the present invention for treatment of PI3K-associated diseases, disorders or conditions. In some embodiments, the one or more additional agents comprises a JAK kinase inhibitor. The one or more additional pharmaceutical agents can be administered to a patient simultaneously or sequentially.

Example antibodies for use in combination therapy include but are not limited to Trastuzumab (e.g. anti-HER2), Ranibizumab (e.g. anti-VEGF-A), Bevacizumab (trade name Avastin, e.g. anti-VEGF, Panitumumab (e.g. anti-EGFR), Cetuximab (e.g. anti-EGFR), Rituxan (anti-CD20) and antibodies directed to c-MET.

In some embodiments, the one or more additional pharmaceutical agents comprises an inhibitor of the IL-17 pathway.

In some embodiments, the one or more additional pharmaceutical agents comprises a calcineurin inhibitor. In some embodiments, the calcineurin inhibitor is cyclosporine, tacrolimus, pimecrolimus, or combinations thereof.

In some embodiments, the one or more additional pharmaceutical agents comprises an additional PI3Kδ inhibitor.

In some embodiments, the one or more additional pharmaceutical agents comprises a PDE4 inhibitor.

In some embodiments, the one or more additional pharmaceutical agents comprises an AHR agonist.

In some embodiments, the one or more additional pharmaceutical agents comprises an inhibitor of the IL-23 pathway.

### Kits

The present disclosure also includes pharmaceutical kits useful, for example, in the treatment or prevention of skin disorders as described herein, which include one or more containers containing one or more pharmaceutical compositions of the present disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Examples 1A-1D. Diastereoisomers of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one

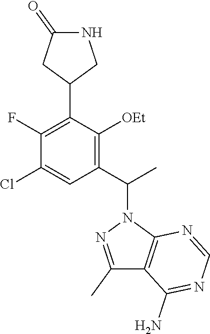 [SMILES: CCOc1c(C(C)n2nc(C)c3c(N)ncnc32)cc(Cl)c(F)c1C1CNC(=O)C1]

### Step 1. 1-(5-Chloro-2-ethoxy-3-iodo-4-methylphenyl)ethanol

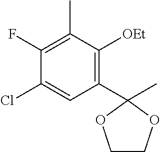 [SMILES: CCOc1c(C2(C)OCCO2)cc(Cl)c(F)c1C]

A solution of 1-(5-chloro-2-ethoxy-4-fluoro-3-iodophenyl)ethanone (20.0 g, 58.4 mmol; see e.g., U.S. Pat. No. 9,199,982, Example 212, step 1) and 1,2-ethanediol (6.5 mL, 120 mmol) in toluene (190 mL) was treated with p-toluenesulfonic acid monohydrate (1.1 g, 5.8 mmol). The flask was fitted with a Dean-Stark trap that was filled with sieves, and refluxed for 3 h. The reaction mixture was cooled and added to ice cooled saturated sodium bicarbonate solution (250 mL) and extracted with ethyl acetate. The organic layer was washed with brine, dried over sodium sulfate, filtered, and concentrated to a crude orange oil. The crude material was purified by flash column chromatography using ethyl acetate in hexanes (0%-20%) to give the desired product (22 g, 99%). LCMS for C₁₂H₁₄ClFIO₃ (M+H)⁺: m/z=387.0; Found: 386.9.

### Step 2. Ethyl (2E)-3-[3-chloro-6-ethoxy-2-fluoro-5-(2-methyl-1,3-dioxolan-2-yl)phenyl]acrylate

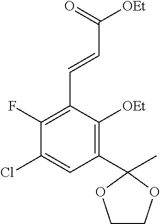 [SMILES: CCOC(=O)/C=C/c1c(F)c(Cl)cc(C2(C)OCCO2)c1OCC]

A mixture of 2-(5-chloro-2-ethoxy-4-fluoro-3-iodophenyl)-2-methyl-1,3-dioxolane (22 g, 58 mmol) (from Step 1), ethyl (2E)-3-(4,4,5,5-tetramethyl-1,3,2-dioxaborolan-2-yl)acrylate (16 mL, 70 mmol), and potassium carbonate (24 g, 170 mmol) in 1,4-dioxane (230 mL) and water (110 mL) was degassed with nitrogen for 10 min. The reaction mixture was treated with [1,1′-bis (diphenylphosphino)ferrocene]dichloropalladium(II),complex with dichloromethane (1:1) (2.4 g, 2.9 mmol), degassed with nitrogen for another 10 min, and heated at 80° C. for 2 h. The reaction mixture was filtered through Celite and washed with ethyl acetate (300 mL). The filtrate was poured into water (400 mL). The aqueous layer was separated and extracted with additional ethyl acetate (300 mL). The combined organic extracts were washed with brine, dried over sodium sulfate, filtered, and concentrated to a crude brown solid. The crude material was purified by flash column chromatography using ethyl acetate in hexanes (0%-30%) to give the desired product (20 g, 96%). ¹H NMR (400 MHz, CDCl₃) δ 7.74 (d, J=16.5 Hz, 1H), 7.56 (d, J=8.6 Hz, 1H), 6.70 (dd, J=16.5, 0.9 Hz, 1H), 4.26 (q, J=7.1 Hz, 2H), 4.10-3.99 (m, 2H), 3.91 (q, J=7.0 Hz, 2H), 3.87-3.76 (m, 2H), 1.73 (s, 3H), 1.44 (t, J=7.0 Hz, 3H), 1.33 (t, J=7.1 Hz, 3H). LCMS for C₁₇H₂₁ClFO₅ (M+H)⁺: m/z=359.1; Found: 359.1.

### Step 3. Ethyl 3-[3-chloro-6-ethoxy-2-fluoro-5-(2-methyl-1,3-dioxolan-2-yl)phenyl]-4-nitrobutanoate

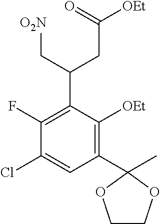 [SMILES: CCOC(=O)CC(C[N+](=O)[O-])c1c(F)c(Cl)cc(C2(C)OCCO2)c1OCC]

A solution ethyl (2E)-3-[3-chloro-6-ethoxy-2-fluoro-5-(2-methyl-1,3-dioxolan-2-yl)phenyl]acrylate (10 g, 28 mmol) (from Step 2) in nitromethane (100 mL) was treated with 1,8-diazabicyclo[5.4.0]undec-7-ene (4.6 mL, 31 mmol) and stirred at 60° C. for 15 h. The reaction mixture was poured into water (400 mL) and extracted with ethyl acetate (2×300 mL). The combined organic extracts were washed with brine, dried over sodium sulfate, filtered, and concentrated to a crude orange oil. The crude material was purified by flash column chromatography using ethyl acetate in hexanes (0%-30%) to give the desired product as a mixture of enantiomers (10.4 g, 89%). ¹H NMR (400 MHz, CDCl₃) δ 7.52 (d, J=9.1 Hz, 1H), 4.82 (ddd, J=12.5, 7.6, 1.4 Hz, 1H), 4.68 (dd, J=12.5, 7.2 Hz, 1H), 4.54-4.40 (m, 1H), 4.15-3.90 (m, 6H), 3.89-3.75 (m, 2H), 2.85 (ddd, J=16.0, 8.6, 1.4 Hz, 1H), 2.73 (dd, J=16.1, 6.2 Hz, 1H), 1.70 (s, 3H), 1.47 (t, J=7.0 Hz, 3H), 1.21 (t, J=7.1 Hz, 3H). LCMS for C₁₈H₂₄ClFNO₇ (M+H)⁺: m/z=420.1; Found: 420.1.

### Step 4. Enantiomers 4-[3-chloro-6-ethoxy-2-fluoro-5-(2-methyl-1,3-dioxolan-2-yl)phenyl]pyrrolidin-2-one

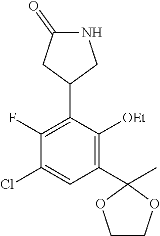 [SMILES: CCOc1c(C2(C)OCCO2)cc(Cl)c(F)c1C1CNC(=O)C1]

A suspension of ethyl 3-[3-chloro-6-ethoxy-2-fluoro-5-(2-methyl-1,3-dioxolan-2-yl)phenyl]-4-nitrobutanoate (1.0 g, 2.4 mmol) (from Step 3) in ethanol (16 mL) was warmed to dissolve the solid. The solution was cooled back to ambient temperature, degassed with nitrogen, and treated with a slurry of 2800 Raney Nickel in water (1.5 mL). The reaction mixture was degassed again with nitrogen and hydrogenated with a balloon of hydrogen for 3 h. The reaction mixture was filtered through Celite and concentrated to give the intermediate amino ester (0.93 g, 100%). The intermediate amino ester was dissolved in toluene (12 mL) and heated at 110° C. for 12 h. The reaction mixture was cooled to ambient temperature, at which point a solid precipitated from solution. This mixture was cooled to 0° C., stirred for 30 min, filtered, washed with cold toluene, and dried to give the desired product as a mixture of enantiomers (0.61 g, 75%). LCMS for C₁₆H₂₀ClFNO₄ (M+H)⁺: m/z=344.1; Found: 344.1. The mixture of enantiomers was separated by chiral HPLC to give the individual enantiomers as peak 1 and peak 2 (RT=5.39 min and 7.01 min, respectively; Phenomenex Lux Cellulose C-1, 21.2×250 mm, 5 micron particle size, eluting with 20% ethanol in hexanes at 18 mL/min).

### Step 5. Enantiomers of 4-(3-acetyl-5-chloro-2-ethoxy-6-fluorophenyl)pyrrolidin-2-one

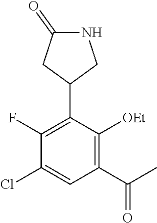 [SMILES: CCOc1c(C(C)=O)cc(Cl)c(F)c1C1CNC(=O)C1]

The separated enantiomers from step 4 were each processed individually to the final compounds. A solution of 4-[3-chloro-6-ethoxy-2-fluoro-5-(2-methyl-1,3-dioxolan-2-yl)phenyl]pyrrolidin-2-one (1.7 g, 5.0 mmol) (from Step 4) in methanol (17 mL) was treated with 6.0 M hydrogen chloride in water (11 mL, 69 mmol) dropwise and stirred 20° C. for 30 min. The reaction mixture was added dropwise to ice cooled saturated sodium bicarbonate solution (75 ml) and extracted with ethyl acetate (2×100 ml). The combined organic extracts were washed with brine, dried over sodium sulfate, filtered, and concentrated to give the desired products [from peak 1 (1.5 g, 99%); from peak 2 (1.5 g, 99%)] that were used without further purification. From peak 1: ¹H NMR (400 MHz, DMSO-d₆) δ 7.84 (s, 1H), 7.70 (d, J=8.6 Hz, 1H), 4.16-3.99 (m, 1H), 3.83 (q, J=7.0 Hz, 2H), 3.65-3.54 (m, 1H), 3.30-3.23 (m, 1H), 2.55 (s, 3H), 2.33 (dd, J=16.8, 8.4 Hz, 1H), 1.30 (t, J=7.0 Hz, 3H). LCMS for C₁₄H₁₆ClFNO₃ (M+H)⁺: m/z=300.1; Found: 300.0. From peak 2: ¹H NMR (400 MHz, DMSO-d₆) δ 7.84 (s, 1H), 7.70 (d, J=8.6 Hz, 1H), 4.13-4.00 (m, 1H), 3.87-3.77 (m, 2H), 3.65-3.55 (m, 1H), 3.31-3.23 (m, 1H), 2.55 (s, 3H), 2.32 (ddd, J=16.9, 8.4, 1.6 Hz, 1H), 1.30 (t, J=7.0 Hz, 3H). LCMS for C₁₄H₁₆ClFNO₃ (M+H)⁺: m/z=300.1; Found: 300.1.

### Step 6. Diastereoisomers of 4-[3-chloro-6-ethoxy-2-fluoro-5-(1-hydroxyethyl)phenyl]pyrrolidin-2-one

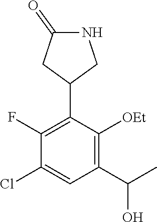 [SMILES: CCOc1c(C(C)O)cc(Cl)c(F)c1C1CNC(=O)C1]

The enantiomers from step 5 were each processed individually to the final products. A solution of 4-(3-acetyl-5-chloro-2-ethoxy-6-fluorophenyl)pyrrolidin-2-one (0.402 g, 1.34 mmol) (from Step 5) in anhydrous methanol (6.7 mL) under an atmosphere of nitrogen at 0° C. was treated with sodium tetrahydroborate (0.10 g, 2.7 mmol) and stirred at 0° C. for 30 min. The reaction mixture was quenched with water at 0° C. and poured into water (50 mL)/ethyl acetate (100 mL) while stirring. The mixture was warmed to ambient temperature and the aqueous layer was separated and extracted with additional ethyl acetate (50 mL). The combined organic extracts were washed with brine, dried over sodium sulfate, filtered, and concentrated to give white foams. The crude material were purified by flash column chromatography using acetonitrile (containing 7% methanol) in dichloromethane (0%-100%) to give the desired products as mixtures of diastereoisomers [from peak 1 (0.40 g, 99%); from peak 2 (0.40 g, 99%)]. From peak 1: LCMS for C₁₄H₁₈ClFNO₃ (M+H)⁺: m/z=302.1; Found: 302.0. From peak 2: LCMS for C₁₄H₁₈ClFNO₃ (M+H)⁺: m/z=302.1; Found: 302.1.

### Step 7. Diastereoisomers of 4-[3-chloro-5-(I-chloroethyl)-6-ethoxy-2-fluorophenyl]pyrrolidin-2-one

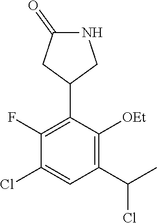 [SMILES: CCOc1c(C(C)Cl)cc(Cl)c(F)c1C1CNC(=O)C1]

The mixture of diastereoisomers from step 6 were each processed individually to the final products. A solution of 4-[3-chloro-6-ethoxy-2-fluoro-5-(1-hydroxyethyl)phenyl]pyrrolidin-2-one (0.41 g, 1.4 mmol) (from Step 6) in methylene chloride (12 mL) was treated with N,N-dimethylformamide (0.011 mL, 0.14 mmol) followed by thionyl chloride (0.21 mL, 2.9 mmol) dropwise and stirred at 20° C. for 30 min. The reaction mixture was added dropwise to ice cooled saturated sodium bicarbonate solution and extracted with dichloromethane. The organic layer was separated and washed with brine, dried over sodium sulfate, filtered, and concentrated to give the desired products [from peak 1 (0.38 g, 87%); from peak 2 (0.39 g, 89%)] along with 17-18% of the styrene that formed from chloride elimination. These mixtures were used without further purification. From peak 1: LCMS for C₁₄H₁₇Cl₂FNO₂ (M+H)⁺: m/z=320.1; Found: 320.0. From peak 2: LCMS for C₁₄H₁₇Cl₂FNO₂ (M+H)⁺: m/z=320.1; Found: 320.0.

### Step 8. Diastereoisomers of 4-{3-[1-(4-amino-3-methyl-1H-pyrazolo[3,4-d]pyrimidin-1 -yl)ethyl]-5-chloro-2-ethoxy-6-fluorophenyl}pyrrolidin-2-one

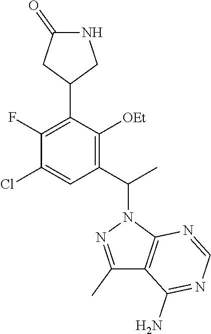 [SMILES: CCOc1c(C(C)n2nc(C)c3c(N)ncnc32)cc(Cl)c(F)c1C1CNC(=O)C1]

The mixture of diastereoisomers from step 7 were each processed individually to the final products. A mixture of 4-[3-chloro-5-(1-chloroethyl)-6-ethoxy-2-fluorophenyl]pyrrolidin-2-one (0.36 g, 1.1 mmol) (from Step 7), 3-methyl-1H-pyrazolo[3,4-d]pyrimidin-4-amine (0.19 g, 1.3 mmol), cesium carbonate (0.54 g, 1.7 mmol) and potassium iodide (18 mg, 0.11 mmol) in N,N-dimethylformamide (7.4 mL) was heated at 100° C. for 4.5 h. The reaction mixture was poured into water (30 ml) and extracted with ethyl acetate (3×50 mL) to give a mixture of diastereoisomers ((S)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one; (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one; (S)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one; and (R)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one). The mixture of diastereoisomers were purified by preparative LCMS (XBridge C18 column, eluting with a gradient of acetonitrile/water containing 0.1% ammonium hydroxide, at flow rate of 60 mL/min) to give the desired products [from peak 1 were isolated peak A (Example 1A) (0.13 g, 54%) and peak B (Example 1B) (0.11 g, 46%); from peak 2 were isolated peak A (Example 1C) (0.15 g, 63%) and peak B (Example 1D) (0.14 g, 55%)]. Example 1B: ¹H NMR (300 MHz, DMSO-d₆) δ 8.12 (s, 1H), 7.82 (s, 1H), 7.52 (d, J=8.5 Hz, 1H), 7.30 (br s, 1H), 6.23 (q, J=7.0 Hz, 1H), 4.05-3.90 (m, 1H), 3.88-3.78 (m, 2H), 3.63-3.53 (m, 1H), 3.29-3.20 (m, 1H), 2.54 (s, 3H), 2.38-2.21 (m, 1H), 1.70 (d, J=7.1 Hz, 3H), 1.39 (t, J=6.9 Hz, 3H). LCMS for C₂₀H₂₃ClFN₆O₂ (M+H)⁺: m/z=433.2; Found: 433.1. Example 1C: ¹H NMR (500 MHz, DMSO-d₆) δ 8.12 (s, 1H), 7.77 (s, 1H), 7.53 (d, J=8.5 Hz, 1H), 7.26 (br s, 2H), 6.24 (q, J=7.0 Hz, 1H), 4.04-3.94 (m, 1H), 3.93-3.85 (m, 1H), 3.84-3.77 (m, 1H), 3.61-3.53 (m, 1H), 3.27-3.22 (m, 1H), 2.54 (s, 3H), 2.30 (dd, J=18.1, 8.6 Hz, 1H), 1.71 (d, J=7.1 Hz, 3H), 1.40 (t, J=6.9 Hz, 3H). LCMS for C₂₀H₂₃ClFN₆O₂(M+H)⁺: m/z=433.2; Found: 433.1.

### Example 2. (R)-4-(3-((S)-1-(4-Amino-3-methyl-1H-pyrazolo[3,4-d]pyrimidin-1-yl)ethyl)-5-chloro-2-ethoxy-6-fluorophenyl)pyrrolidin-2-one Hydrochloric Acid Salt

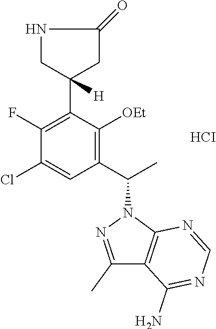

### Step 1. (R)-1-(5-chloro-2-ethoxy-4-fluoro-3-((R)-5-oxopyrrolidin-3-yl)phenyl)ethyl Methanesulfonate

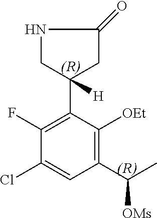

(R)-4-(3-Chloro-6-ethoxy-2-fluoro-5-((R)-1-hydroxyethyl)phenyl)pyrrolidin-2-one (see e.g., U.S. Pat. No. 10,336,759, compound xiii; 172.0 g, 570.0 mmol) (consisted of 147 g at 99.83%: 0.09% chiral purity, 99.33% chemical purity; and 25 g, 87.46%: 12.54% chiral purity, 86.74% chemical purity) was dissolved in methylene chloride (860 mL). N,N-diisopropylethylamine (149 mL, 855 mmol) was added to the solution at from about −7° C. to about 2° C. Methanesulfonyl chloride (57.4 mL, 741 mmol) was added dropwise to the reaction mixture over 25 min. The suspension turned into a clear solution. At 30 min reaction time point HPLC indicated the reaction was complete. This reaction mixture containing (R)-1-(5-chloro-2-ethoxy-4-fluoro-3-((R)-5-oxopyrrolidin-3-yl)phenyl)ethyl methanesulfonate was used directly in the next reaction.

### Step 2. (R)-4-(3-chloro-6-ethoxy-2-fluoro-5-((S)-1-hydrazinylethyl)phenyl)pyrrolidin-2-one

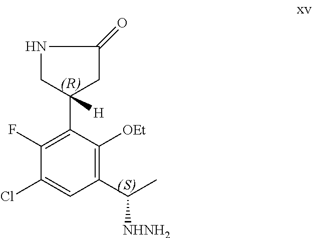

At 0° C., hydrazine (178.9 mL, 5.7 mol) was added in one portion followed by N-methylpyrrolidinone (860 mL) to the reaction mixture containing (R)-1-(5-chloro-2-ethoxy-4-fluoro-3-((R)-5-oxopyrrolidin-3-yl)phenyl)ethyl methanesulfonate from Step 1. The reaction mixture turned cloudy and some precipitates formed. The mixture was heated to 40-57° C. under nitrogen for 90 min. HPLC indicated all the mesylate had been consumed. The reaction mixture was cooled to room temperature and a saturated solution of sodium bicarbonate (28.3 g) in water (300 mL) was added. The mixture was stirred for 20 min, at which time dichloromethane (300 mL) was added. The organic layer was separated and stirred with a solution of sodium bicarbonate (14.2 g) in water (150 mL). The aqueous layer was extracted with dichloromethane (200 mL×2). The combined organic layers were washed with brine (80 mL), dried over anhydrous Na₂SO₄ (311 g), concentrated, and azeotroped with toluene (250 mL) to give a colorless N-methylpyrrolidinone solution containing (R)-4-(3-chloro-6-ethoxy-2-fluoro-5-((S)-1-hydrazinylethyl)phenyl)pyrrolidin-2-one which was used directly in the next reaction. A sample was purified for NMR analysis. ¹H NMR (400 MHz, DMSO-d₆), δ 7.88 (s, 1H), 7.66 (d, J=8.5 Hz, 1H), 4.42 (q, J=6.7 Hz, 1H), 4.06-3.88 (m, 2H), 3.79-3.66 (m, 1H), 3.65-3.51 (m, 1H), 3.24 (t, J=8.8 Hz, 1H), 2.60-2.46 (m, 1H), 2.36-2.25 (m, 1H), 1.37 (t, J=6.9 Hz, 3H), 1.26 (d, J=6.8 Hz, 3H). LCMS for C₁₄H₁₉ClFN₃O₂(M+H)⁺: m/z=316.1.

### Step 3. 5-Amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile

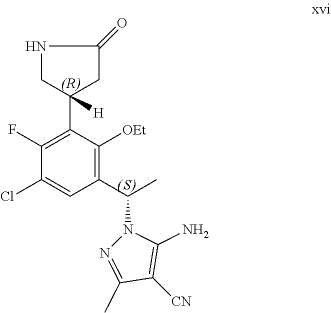

With stirring, (1-ethoxyethylidene)malononitrile (101 g, 741 mmol) was added to the N-methylpyrrolidinone solution of (R)-4-(3-chloro-6-ethoxy-2-fluoro-5-((S)-1-hydrazinylethyl)phenyl)pyrrolidin-2-one from Step 2, in portions and the mixture was stirred at room temperature under nitrogen. After 15 min, HPLC analysis indicated 11% starting material hydrazine, (R)-4-(3-chloro-6-ethoxy-2-fluoro-5-((S)-1-hydrazinylethyl)phenyl)pyrrolidin-2-one, relative to product 5-amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile. N,N-Diisopropylethylamine (15 mL, 86 mmol) was added and the reaction mixture was stirred at room temperature for 15 h. HPLC analysis indicated 5.6% of starting material remained. N,N-Diisopropylethylamine (5 mL, 30 mmol) was added and the reaction mixture was stirred at room temperature for 5 h. HPLC indicated 5.6% starting material remained. The reaction mixture was stirred for 2.5 days and combined with two similar batches and worked up together.

The reaction mixtures of three batches of 5-amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile were combined. An aqueous 0.5 M sodium hydroxide solution (3.8 L) was added at 10-20° C. and stirred for 5 min. HPLC indicated that all starting material (1-ethoxyethylidene)malononitrile was consumed. Ethyl acetate (4.0 L) was added and the mixture was stirred for 15 min. The layers were separated. The organic layer was washed with 0.5 M sodium hydroxide in water (2.38 L). The layers were separated. The combined aqueous layer was extracted with ethyl acetate (2×2 L). The combined organic layers were washed with 1.0 M aqueous hydrochloric acid (3.56 L) and the pH of the resulting aqueous layer was 2-3. The organic layer was washed with brine (5 L), dried over anhydrous Na₂SO₄, concentrated, and dried under high vacuum for 40 h to give 5-amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile as a light brown foamy solid (702.7 g). ¹H NMR (500 MHz, DMSO-d₆) δ 7.78 (s, 1H), 7.44 (d, J=8.4 Hz, 1H), 6.53 (s, 2H), 5.64 (q, J=6.7 Hz, 1H), 3.96 (m, 1H), 3.74 (m, 1H), 3.34 (m, 1H), 3.58 (m, 2H), 2.59-2.50 (m, 1H), 2.29 (m, 1H), 2.04 (s, 3H), 1.57 (d, J=6.8 Hz, 3H), 1.37 (t, J=6.9 Hz, 3H). LCMS for C₁₉H₂₂ClFN₅O₂(M+H)⁺: m/z=406.1.

The overall yield of 5-amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile over three steps (mesylation, hydrazinolysis and pyrazole formation) was calculated to be 72.8% from the total input of (R)-4-(3-chloro-6-ethoxy-2-fluoro-5-((R)-1-hydroxyethyl)phenyl)pyrrolidin-2-one. The purity was determined by HPLC to be about 80%. HPLC analysis indicated some product existing in the basic aqueous layer which was subsequently extracted with EtOAc (2 L), washed with 1.0 M aqueous hydrochloric acid and brine, dried with anhydrous sodium sulfate, concentrated, and dried on high vacuum pump for 40 h to afford 5-amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile as a brown oil (134 g, 13.9%).

### Step 4. (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one

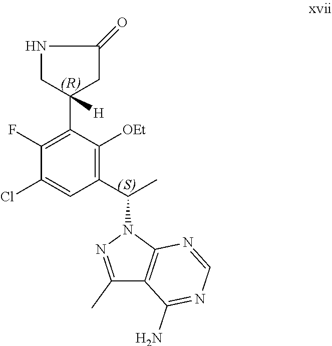

5-Amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile (702.7 g, 1731 mmol) was added to a reaction vessel with formamidine acetate (1802 g, 17.31 mol) and 1,2-ethanediol (3.51 L). The reaction mixture was heated at 102-103° C. with stirring for 18 h. The reaction mixture was cooled to room temperature and ethyl acetate (7 L) and water (6 L) were added and the biphasic mixture was stirred for 15 min. The organic layer was separated and the aqueous layer was diluted with additional water (4.5 L) and ethyl acetate (3 L) and stirred for 10 min. The organic layer was separated. The aqueous layer was further extracted with ethyl acetate (2 L). The organic layers were combined and stirred with water (4.5 L). The aqueous layer was separated and the organic layer was filtered through a pad of celite (about 1 kg). The organic layer was extracted with 1.0 M aqueous hydrochloric acid (7 L) by stirring the mixture for 10 min. The aqueous layer was separated. The clear brown organic layer was stirred with additional 1.0 M aqueous hydrochloric acid (3 L) for 10 min. The aqueous layer was separated. The aqueous acidic layers were combined and washed with toluene (500 mL). The aqueous acidic solution was cooled with an ice-water bath and methylene chloride (4 L) was added. At 5-15° C., a solution of sodium hydroxide (530 g) in water (530 mL) (50% NaOH solution) was added slowly until to a solution pH of 11-12. Solid precipitates were observed. Additional methylene chloride (3.5 L) and methanol (300 mL) were added and the mixture was stirred for 10-15 min. The solid product was collected by filtration and dried on the filter under suction for 16 h to give (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one (289.7 g) as a brown solid. ¹H NMR (400 MHz, DMSO-d₆) δ 8.11 (s, 1H), 7.82 (s, 1H), 7.52 (d, J=8.5 Hz, 1H), 7.30 (br s, 2H), 6.23 (q, J=7.0 Hz, 1H), 3.97 (p, J=9.2 Hz, 1H), 3.90-3.73 (m, 2H), 3.57 (t, J=9.9 Hz, 1H), 3.25 (dd, J=9.2, 8.7 Hz, 1H), 2.48 (s, 3H), 2.60-2.50 (m, 1H), 2.36-2.20 (m, 1H), 1.69 (d, J=7.1 Hz, 3H), 1.39 (t, J=6.9 Hz, 3H). LCMS for C₂₀H₂₃ClFN₆O₂(M+H)+: m/z=433.3.

The filtrate was transferred into a separatory funnel and the organic layer was separated. The aqueous layer was stirred with methylene chloride (5 L) and methanol (200 mL). The combined organic layer was dried over anhydrous sodium sulfate, concentrated, dried on high vacuum pump for 16 h to give additional amount 259.3 g as a brown solid. The total yield of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one was 548.3 g in 73.2% yield.

### Step 5. (R)-4-(3-((S)-1-(4-Amino-3-methyl-1H-pyrazolo[3,4-d]pyrimidin-1-yl)ethyl)-5-chloro-2-ethoxy-6-fluorophenyl)pyrrolidin-2-one Hydrochloride Salt

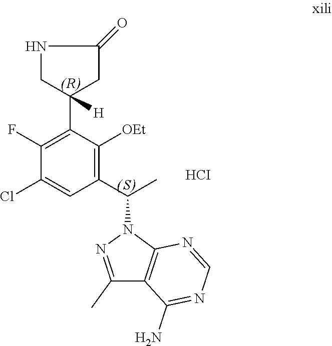

A 1.0 M aqueous hydrochloric acid (HCl, 5.0 L, 5.0 mol) solution was added to (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one (609.8 g, 1.409 mol) at room temperature. The resulting thick slurry was then heated to 50° C. to afford a clear solution. An additional 1.82 L of 1.0 M aqueous hydrochloric acid solution (HCl, 1.82 L, 1.82 mol; total 6.82 L, 6.82 mol, 4.84 equiv) was added to the clear solution at 50° C. and the solution was then filtered through a polish filter at approximately 50° C. The polish filtered reaction mixture was gradually cooled to room temperature over 2 h before it was further cooled to 0-5° C. The reaction mixture was stirred at 0-5° C. for at least 20 min to initiate precipitation. The resulting solids were collected by filtration, rinsed with a portion of cold mother liquor, followed by 1.0 M aqueous hydrochloric acid (HCl, 200 mL), and dried on the filter funnel at room temperature under suction to constant weight (in about 39 h) to afford the hydrochloric acid salt of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one (348.7 g, 661.2 g theoretical, 52.7%) as white crystalline powder. ¹H NMR (400 MHz, DMSO-d₆) δ 9.39 (br s, 1H), 9.05 (br s, 1H), 8.50 (s, 1H), 7.84 (s, 1H), 7.59 (d, J=8.4 Hz, 1H), 6.28 (q, J=6.9 Hz, 1H), 3.95 (m, 1H), 3.79 (m, 2H), 3.55 (m, 1H), 3.22 (m, 1H), 2.59 (s, 3H), 2.55 (ddd, J=16.8, 10.3, 2.3 Hz, 1H), 2.28 (ddd, J=16.8, 8.6, 1.5 Hz, 1H), 1.73 (d, J=7.0 Hz, 3H), 1.38 (t, J=6.9 Hz, 3H) ppm. ¹³C NMR (100 MHz, DMSO-d₆) δ 175.3, 156.4 (J_{CF}=249.8 Hz), 153.8 (J_{CF}=7.0 Hz), 152.4, 150.8, 147.3, 144.3, 131.4 (J_{CF}=3.5 Hz), 127.3, 126.4 (J_{CF}=12.6 Hz), 116.1 (J_{CF}=18.4 Hz), 98.0, 72.1, 49.1, 46.6, 36.0, 29.4, 21.0, 15.4, 14.6 ppm. ¹⁹F NMR (376 MHz, DMSO-d₆) δ −113.6 (d, J_{FH}=7.7 Hz) ppm. C₂₀H₂₃Cl₂FN₆O₂(MW 469.34); LCMS (EI) m/e 433.2 (M⁺+H; exact mass: 432.15). Water content by KF: 3.63% by weight; Chloride (Cl⁻) content by titration: 7.56% by weight (7.56% by theory). Representative DSC, TGA, and X-Ray Powder Diffraction data can be found, for example, in U.S. Pat. No. 10,336,759.

### Example 3. Alternative Synthesis of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one Hydrochloride

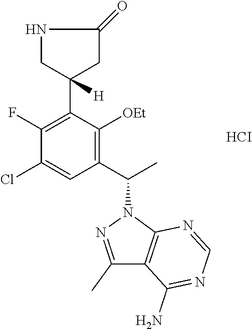 [SMILES: CCOc1c([C@H](C)n2nc(C)c3c(N)ncnc32)cc(Cl)c(F)c1[C@@H]1CNC(=O)C1 ; "HCI"]

### Step 1. (R)-4-(3-acetyl-5-chloro-2-ethoxy-6-fluorophenyl)pyrrolidin-2-one (xix)

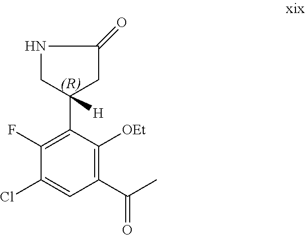

(4R)-4-[3-Chloro-6-ethoxy-2-fluoro-5-(1-hydroxyethyl)phenyl]pyrrolidin-2-one (as a mixture of two diastereomers with R-configuration at the pyrrolidinone and R- or S-configurations at the secondary alcohol) (see e.g., U.S. Pat. No. 10,336,759, compound xiii, 16.7 g, 55.3 mmol) was dissolved in dichloromethane (167 mL). The solution was cooled in an ice-water bath and Dess-Martin periodinane (35.2 g, 83.0 mmol) was added in small portions. The reaction mixture was stirred at room temperature for 2 h, at which time HPLC analysis showed reaction completion. A solution of sodium sulfite (28 g, 220 mmol) in water (70 mL) was added to the reaction mixture and the mixture was stirred for 20 min. A 1.0 M sodium hydroxide solution was added to the mixture and stirred for 10 min. The layers were allowed to settle and the organic layer was separated and washed sequentially with 1 M aqueous sodium hydroxide solution (66 mL) and water (60 mL). The organic layer was dried over anhydrous sodium sulfate. The drying agent was removed by filtration and the filtrate was concentrated to give (R)-4-[3-acetyl-5-chloro-2-ethoxy-6-fluorophenyl]pyrrolidin-2-one as an oil which was used in the next reaction without further purification.

### Step 2. (R,E)-tert-butyl 2-(1-(5-chloro-2-ethoxy-4-fluoro-3-(5-oxopyrrolidin-3-yl)phenyl)ethylidene)hydrazinecarboxylate

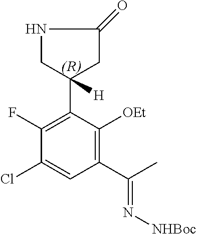

Crude (R)-4-[3-acetyl-5-chloro-2-ethoxy-6-fluorophenyl]pyrrolidin-2-one (from Step 1) was dissolved in methanol (60 mL) and t-butyl carbazate (8.04 g, 60.8 mmol) was added to the solution. The reaction mixture was stirred at 65° C. for 3.5 days, at which time HPLC analysis showed reaction completion. The mixture was concentrated under reduced pressure and the residue was purified by silica gel chromatography eluting with a mixture of 0-5% of methanol in ethyl acetate to give (R,E)-tert-butyl 2-(1-(5-chloro-2-ethoxy-4-fluoro-3-(5-oxopyrrolidin-3-yl)phenyl)ethylidene)hydrazinecarboxylate (19.5 g, 85%). ¹H NMR (500 MHz, DMSO-d₆) δ 9.83 (s, 1H), 7.78 (s, 1H), 7.36 (d, J=8.6 Hz, 1H), 4.07 (p, J=9.1 Hz, 1H), 3.84-3.69 (m, 2H), 3.59 (t, J=9.5 Hz, 1H), 3.28 (t, J=9.5 Hz, 1H), 2.54 (m, 1H), 2.33 (m, 1H), 2.14 (s, 3H), 1.46 (s, 9H), 1.25 (t, J=7.0 Hz, 3H). LCMS for C₁₉H₂₅ClFN₃NaO₄ (M+Na)⁺: m/z=436.1.

### Step 3. Tert-Butyl 2-((S)-1-(5-chloro-2-ethoxy-4-fluoro-3-((R)-5-oxopyrrolidin-3-yl)phenyl)ethyl)hydrazinecarboxylate

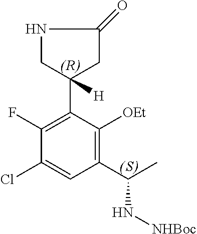

(R,E)-tert-butyl 2-(1-(5-chloro-2-ethoxy-4-fluoro-3-(5-oxopyrrolidin-3-yl)phenyl)ethylidene)hydrazinecarboxylate (0.5 g, 1.2 mmol) was dissolved in methanol (25 mL) and the solution was bubbled with nitrogen gas for 5 min. Bis(1,5-cyclooctadiene)rhodium(I)tetrafluoroborate (35 mg, 0.086 mmol) and (R)-(−)-1-{(S)-2-[bis(4-trifluoromethylphenyl)phosphine]ferrocenyl}ethyl-di-t-butylphosphine (64 mg, 0.094 mmol) were added to the solution and the resulting reaction mixture was bubbled with nitrogen gas for 30 min. The reaction mixture was then agitated under hydrogen gas (56 psi) pressure for 2.5 days. The reaction mixture was concentrated under reduced pressure and the resulting residue was purified by silica gel column chromatography eluting with a mixture of methanol (0-10%) in ethyl acetate. The desired fractions were concentrated to give tert-butyl 2-((S)-1-(5-chloro-2-ethoxy-4-fluoro-3-((R)-5-oxopyrrolidin-3-yl)phenyl)ethyl)hydrazinecarboxylate (428 mg, 85% yield). ¹H NMR (500 MHz, DMSO-d₆) δ 8.18 (s, 1H), 7.78 (s, 1H), 7.53 (d, J=8.2 Hz, 1H), 4.73 (s, 1H), 4.41 (br s, 1H), 3.98 (m, 1H), 3.75 (m, 2H), 3.61 (m, 1H), 3.26 (m, 1H), 2.53 (m, 1H), 2.29 (dd, J=17.6, 8.6 Hz, 1H), 1.32 (s, 12H), 1.10 (d, J=6.5 Hz, 1H). LCMS for C₁₉H₂₇ClFN₃NaO₄ (M+Na)⁺: m/z=437.9. Chiral HPLC analysis indicated the product contained the desired diastereomer tert-butyl-2-((S)-1-(5-chloro-2-ethoxy-4-fluoro-3-((R)-5-oxopyrrolidin-3-yl)phenyl)ethyl)hydrazine carboxylate at 85.6% and the undesired diastereomer tert-butyl-2-((R)-1-(5-chloro-2-ethoxy-4-fluoro-3-((R)-5-oxopyrrolidin-3-yl)phenyl)ethyl)hydrazinecarboxylate at 14.3%.

### Step 4. 5-Amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile

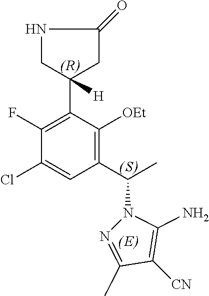

tert-Butyl 2-((S)-1-(5-chloro-2-ethoxy-4-fluoro-3-((R)-5-oxopyrrolidin-3-yl)phenyl)ethyl)hydrazinecarboxylate (130 mg, 0.31 mmol) and p-toluenesulfonic acid monohydrate (86 mg, 0.45 mmol) were added to ethanol (3 mL) and the reaction mixture was heated at 50° C. for 20 h. HPLC analysis showed there was about 88% of unreacted starting material. Additional amount of p-toluene sulfonic acid (86 mg, 0.45 mmol) was charged and the reaction mixture was heated to 60° C. for 24 h. HIPLC analysis showed complete Boc-deprotection. This reaction mixture was added with (1-ethoxyethylidene)malononitrile (61 mg, 0.45 mmol) and N,N-diisopropylethylamine (260 μL, 1.5 mmol). The reaction mixture was stirred at room temperature for 2 h. HIPLC showed completion of pyrazole-ring formation. 1.0 M aqueous sodium hydroxide solution was added to the reaction mixture and stirred for 20 min. Ethyl acetate (20 mL) was added to the mixture and stirred. The biphasic mixture was allowed to settle. The ethyl acetate layer was collected and the aqueous layer was extracted with ethyl acetate (10 mL). The combined ethyl acetate solution was added with 1M aqueous hydrochloric acid (5 mL) and stirred for 15 min. The biphasic mixture was allowed to settle and the organic layer was collected and dried over anhydrous sodium sulfate. Sodium sulfate was removed by filtration and to filtrate was concentrated to give 5-Amino-1-((S)-1-(5-chloro-2-ethoxy-4-fluoro-3-((R)-5-oxopyrrolidin-3-yl)phenyl)ethyl)-3-methyl-1H-pyrazole-4-carbonitrile (126 mg, quantitative yield of crude product) and was used in the next step without further purification.

### Step 5. (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one

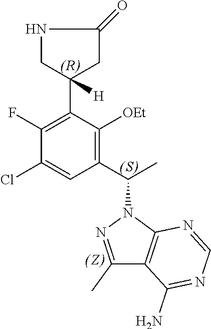

5-Amino-1-{(1S)-1-[5-chloro-2-ethoxy-4-fluoro-3-(5-oxopyrrolidin-3-yl)phenyl]ethyl}-3-methyl-1Hpyrazole-4-carbonitrile (126 mg, 0.31 mmol) was added with formamidine acetate (323 mg, 3.1 mmol) and 1,2-ethanediol (2 mL). The reaction mixture was heated at 104-105° C. with stirring. After 18 h, HPLC analysis showed about 44% of 5-amino-1-{(1S)-1-[5-chloro-2-ethoxy-4-fluoro-3-(5-oxopyrrolidin-3-yl)phenyl]ethyl}-3-methyl-1Hpyrazole-4-carbonitrile remaining. The reaction mixture was heated to 115° C. for 24 h. HPLC analysis showed the reaction was complete. The reaction mixture was cooled to room temperature and ethyl acetate (10 mL) and water (5 ml) were added. The biphasic mixture was stirred. The layers were allowed to separate. The organic layer was collected and the aqueous layer was extracted with ethyl acetate (5 mL). The combined ethyl acetate solution was washed with water (5 mL), dried over anhydrous sodium sulfate. Sodium sulfate was removed by filtration and the filtrate was concentrated to a residue. The residue was purified by silica gel chromatography. The column was eluted with a mixture of methanol (0-5%) in methylene chloride. The desired fractions were combined and evaporated to give (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one (94 mg, 69.9% yield). ¹H NMR (400 MHz, DMSO-d₆) δ 8.11 (s, 1H), 7.82 (s, 1H), 7.52 (d, J=8.5 Hz, 1H), 7.30 (br s, 2H), 6.23 (q, J=7.0 Hz, 1H), 3.97 (p, J=9.2 Hz, 1H), 3.90-3.73 (m, 2H), 3.57 (t, J=9.9 Hz, 1H), 3.25 (dd, J=9.2, 8.7 Hz, 1H), 2.48 (s, 3H), 2.60-2.50 (m, 1H), 2.36-2.20 (m, 1H), 1.69 (d, J=7.1 Hz, 3H), 1.39 (t, J=6.9 Hz, 3H). LCMS for C₂₀H₂₃ClFN₆O₂(M+H)⁺: m/z=433.3.

Chiral HPLC analysis of the product indicated that it contained the desired diastereomer, (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, at 87% and the undesired diastereomer (R)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one at 13%.

### Step 6. (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one Hydrochloride

The title product was prepared according to the procedure described in Example 2, Step 5. The resulting hydrochloride salt matches well with the material made from the synthetic process described in Example 2, in every comparable aspect including chemical purity, chiral purity, and solid state characteristics.

### Example 4. Topical Formulations

Topical formulations were prepared using (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt.

1. A water phase was prepared by mixing water, disodium EDTA, PEG300 NF, and propylene glycol. The mixture was stirred and heated at 65° C. (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt was added and stirred until fully dissolved. PEG300 was found to be more suitable than PEG400 for producing the formulations on a large scale.

2. A xanthan gum phase was prepared by adding xanthan gum into propylene glycol and dispersing at 65° C. Additional propylene glycol (1 g) was used for rinsing.

3. The xanthan gum phase was then added into the water phase and mixed at 65° C. with overhead stirring at 800 rpm.

4. An oil phase was prepared by heating medium chain triglycerides, cetyl alcohol, glyceryl mono- and di-stearate NF, steryl alcohol, light mineral oil NF, white petrolatum USP, and polysorbate 20 NF at 65° C. The mixture was stirred until melted and mixed well.

5. The oil phase was then added into water phase and mixed at 65° C. with overhead stirring at 800 rpm. After mixing well, heating was stopped and the temperature of the resulting oil-in-water emulsion was monitored.

6. Phenoxyethanol was then added to the oil-in-water emulsion when the temperature of the emulsion was 35° C.-40° C.

7. The resulting emulsion was then stirred at a low rate until the emulsion reached at room temperature.

The following formulations in Table 1 below were prepared according to the procedures described above, containing 0% (placebo), 0.003%, 0.005%, 0.01%, 0.050%, 0.1%, 0.5%, and 1.00% w/w (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one on a free base basis. The other formulations in Table 1 are contemplated by the present disclosure.

TABLE 1
Topical Formulations

| | Placebo | 0.003% | 0.005 | 0.01% | 0.05% | 0.10% | 0.5% | 1.0% |
|---|---|---|---|---|---|---|---|---|
| | % w/w | % w/w | % w/w | % w/w | % w/w | % w/w | % w/w | % w/w |
| Compound A HCl* | 0 | 0.00324 | 0.0054 | 0.0108 | 0.054 | 0.108 | 0.54 | 1.08 |
| Purified Water, USP | qs | qs | qs | qs | qs | qs | qs | qs |
| Disodium EDTA, USP | 0.05 | 0.05 | 0.05 | 0.05 | 0.05 | 0.05 | 0.05 | 0.05 |
| PEG 300, NF | 7 | 7 | 7 | 7 | 7 | 7 | 7 | 7 |
| Propylene Glycol, USP | 10 | 10 | 10 | 10 | 10 | 10 | 10 | 10 |
| Xanthan Gum, NF** | 0.35 | 0.35 | 0.35 | 0.35 | 0.35 | 0.35 | 0.35 | 0.35 |
| White Petrolatum, USP | 7 | 7 | 7 | 7 | 7 | 7 | 7 | 7 |
| Light Mineral Oil, NF | 4 | 4 | 4 | 4 | 4 | 4 | 4 | 4 |
| Glyceryl mono and | 3 | 3 | 3 | 3 | 3 | 3 | 3 | 3 |
| distearate, NF | | | | | | | | |
| Cetyl Alcohol, NF | 3 | 3 | 3 | 3 | 3 | 3 | 3 | 3 |
| Stearyl Alcohol, NF | 1.75 | 1.75 | 1.75 | 1.75 | 1.75 | 1.75 | 1.75 | 1.75 |
| Med. Chain | 6 | 6 | 6 | 6 | 6 | 6 | 6 | 6 |
| Triglycerides, NF | | | | | | | | |
| Polysorbate 20, NF | 1.25 | 1.25 | 1.25 | 1.25 | 1.25 | 1.25 | 1.25 | 1.25 |
| Phenoxyethanol, NF | 0.5 | 0.5 | 0.5 | 0.5 | 0.5 | 0.5 | 0.5 | 0.5 |
| Total | 100 | 100 | 100 | 100 | 100 | 100 | 100 | 100 |

*The amount of Compound A HCl is equivalent to the % w/w of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one free base, e.g., 1.08% of HCl is equivalent to 1.0% free base.
**The amount of Xanthan Gum can be varied between 0.3 and 0.4% w/w. Formulations made with both amounts showed good and similar 3-month stability. See Example 5.

### Example 5. Stability of the Topical Formulations

Stability studies on the prepared formulations described in Example 4 were conducted. For these studies, the concentration of Xanthan Gum NF was 0.3% w/w. The studies were carried out at two different conditions, (i) at 40° C./75% relative humidity for 6 weeks, and (ii) at 25° C./60% relative humidity for 4 weeks, 8 weeks, and 12 weeks, respectively. Drug assay was carried out for both conditions at different time points. The results are listed in Table 2 below (amount of Compound A measured in the drug assay is indicated in Table 2). The formulations remained stable over time at the different conditions.

TABLE 2
Assay Stability

| | | 4-week | 6-week | 8-week | 2-week |
|---|---|---|---|---|---|
| | | 25° C. | 40° C. | 25° C. | 25° C. |
| | | 60% RH | 75% RH | 60% RH | 60% RH |
| Drug Assay | | (% of | (% of | (% of | (% of |
| (n = 3) | Time 0 | Comp. A) | Comp. A) | Comp. A) | (Comp. A) |
| Vehicle | 0 | 0 | 0 | 0 | 0 |
| 0.05% | 0.0475 ± 0.001% | 0.0476 ± 0.002% | 0.052 ± 0.003% | 0.049 ± 0.001% | 0.053 ± 0.002% |
| 0.1% | 0.099 ± 0.001% | 0.098 ± 0.001% | 0.102 ± 0.002% | 0.099 ± 0.001% | 0.096 ± 0.002% |
| 0.5% | 0.50 ± 0.01% | 0.49 ± 0.02% | 0.49 ± 0.03% | 0.49 ± 0.01% | 0.48 ± 0.03% |
| 1.0% | 1.05 ± 0.03% | 1.04 ± 0.05% | 0.99 ± 0.01% | 0.98 ± 0.01% | 0.99 ± 0.05% |

The pH of the formulations was also measured. The results are listed in Table 3 below. The pH of the formulations remained stable and did not change over the test period.

TABLE 3
pH Stability

| | | 6-week pH | 12-week pH |
|---|---|---|---|
| % Compound A | | 40° C. | 25° C. |
| HCl | Time 0 pH | 75 RH % | 60 RH % |
| Vehicle | 5.64 | 5.49 | 5.62 |
| 0.05% | 4.55 | 4.36 | 4.60 |
| 0.1% | 4.29 | 4.21 | 4.26 |
| 0.5% | 4.34 | 3.29 | 3.43 |
| 1.0% | 3.11 | 3.08 | 3.16 |

### Example 6: PI3K Enzyme Assay

PI3-Kinase luminescent assay kit including lipid kinase substrate, D-myo-phosphatidylinositol 4,5-bisphosphate (PtdIns(4,5)P2)D (+)-sn-1,2-di-O-octanoylglyceryl, 3-O-phospho linked (PIP2), biotinylated I(1,3,4,5)P4, PI(3,4,5)P3 Detector Protein is purchased from Echelon Biosciences (Salt Lake City, Utah). AlphaScreen™ GST Detection Kit including donor and acceptor beads was purchased from PerkinElmer Life Sciences (Waltham, Mass.). PI3Kδ (p110δ/p85α) is purchased from Millipore (Bedford, Mass.). ATP, MgCl₂, DTT, EDTA, HEPES and CHAPS are purchased from Sigma-Aldrich (St. Louis, Mo.).

### AlphaScreen™ Assay for PI3Kδ

The kinase reaction are conducted in 384-well REMP plate from Thermo Fisher Scientific in a final volume of 40 μL. Inhibitors are first diluted serially in DMSO and added to the plate wells before the addition of other reaction components. The final concentration of DMSO in the assay is 2%. The PI3K assays are carried out at room temperature in 50 mM HEPES, pH 7.4, 5 mM MgCl₂, 50 mM NaCl, 5 mM DTT and CHAPS 0.04%. Reactions are initiated by the addition of ATP, the final reaction mixture consisted of 20 μM PIP2, 20 μM ATP, 1.2 nM PI3Kδ are incubated for 20 minutes. 10 μL of reaction mixture are then transferred to 5 μL 50 nM biotinylated I(1,3,4,5)P4 in quench buffer: 50 mM HEPES pH 7.4, 150 mM NaCl, 10 mM EDTA, 5 mM DTT, 0.1% Tween-20, followed with the addition of 10 μL AlphaScreen™ donor and acceptor beads suspended in quench buffer containing 25 nM PI(3,4,5)P3 detector protein. The final concentration of both donor and acceptor beads is 20 mg/ml. After plate sealing, the plate are incubated in a dark location at room temperature for 2 hours. The activity of the product is determined on Fusion-alpha microplate reader (Perkin-Elmer). IC₅₀ determination is performed by fitting the curve of percent control activity versus the log of the inhibitor concentration using the GraphPad Prism 3.0 software.

### Example 7: PI3K Enzyme Assay

Materials: Lipid kinase substrate, phosphoinositol-4,5-bisphosphate (PIP2), are purchased from Echelon Biosciences (Salt Lake City, Utah). PI3K isoforms α, β, δ and γ are purchased from Millipore (Bedford, Mass.). ATP, MgCl₂, DTT, EDTA, MOPS and CHAPS are purchased from Sigma-Aldrich (St. Louis, Mo.).

The kinase reaction are conducted in clear-bottom 96-well plate from Thermo Fisher Scientific in a final volume of 24 μL. Inhibitors are first diluted serially in DMSO and added to the plate wells before the addition of other reaction components. The final concentration of DMSO in the assay is 0.5%. The PI3K assays are carried out at room temperature in 20 mM MOPS, pH 6.7, 10 mM MgCl₂, 5 mM DTT and CHAPS 0.03%. The reaction mixture is prepared containing 50 μM PIP2, kinase and varying concentration of inhibitors. Reactions are initiated by the addition of ATP containing 2.2 μCi [γ-³³P]ATP to a final concentration of 1000 μM. The final concentration of PI3K isoforms α, β, δ and γ in the assay were 1.3, 9.4, 2.9 and 10.8 nM, respectively. Reactions are incubated for 180 minutes and terminated by the addition of 100 μL of 1 M potassium phosphate pH 8.0, 30 mM EDTA quench buffer. A 100 μL aliquot of the reaction solution are then transferred to 96-well Millipore MultiScreen IP 0.45 μm PVDF filter plate (The filter plate is prewetted with 200 μL 100% ethanol, distilled water, and 1 M potassium phosphate pH 8.0, respectively). The filter plate is aspirated on a Millipore Manifold under vacuum and washed with 18×200 μL wash buffer containing 1 M potassium phosphate pH 8.0 and 1 mM ATP. After drying by aspiration and blotting, the plate is air dried in an incubator at 37° C. overnight. Packard TopCount adapter (Millipore) is then attached to the plate followed with addition of 120 μL Microscint 20 scintillation cocktail (Perkin Elmer) in each well. After the plate sealing, the radioactivity of the product is determined by scintillation counting on Topcount (Perkin-Elmer). IC₅₀ determination is performed by fitting the curve of percent control activity versus the log of the inhibitor concentration using the GraphPad Prism 3.0 software.

The (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt was tested in the assay of Example 7 and determined to be a selective inhibitor for PI3Kδ.

The (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt was tested in the assay of Example 7 and determined to be a >100 fold selective inhibitor for PI3Kδ over each of PI3Kα, PI3Kβ, and PI3Kγ.

### Example 8: PI3KS Scintillation Proximity Assay

Materials

[γ-³³P]ATP (10mCi/mL) was purchased from Perkin-Elmer (Waltham, Mass.).

Lipid kinase substrate, D-myo-Phosphatidylinositol 4,5-bisphosphate (PtdIns(4,5)P2)D (+)-sn-1,2-di-O-octanoylglyceryl, 3-O-phospho linked (PIP2), CAS 204858-53-7, was purchased from Echelon Biosciences (Salt Lake City, Utah). PI3Kδ (p110δ/p85α) was purchased from Millipore (Bedford, Mass.). ATP, MgCl₂, DTT, EDTA, MOPS and CHAPS were purchased from Sigma-Aldrich (St. Louis, Mo.). Wheat Germ Agglutinin (WGA) YSi SPA Scintillation Beads was purchased from GE healthcare life sciences (Piscataway, N.J.).

The kinase reaction was conducted in polystyrene 384-well matrix white plate from Thermo Fisher Scientific in a final volume of 25 μL. Inhibitors were first diluted serially in DMSO and added to the plate wells before the addition of other reaction components. The final concentration of DMSO in the assay was 0.5%. The PI3K assays were carried out at room temperature in 20 mM MOPS, pH 6.7, 10 mM MgCl₂, 5 mM DTT and CHAPS 0.03%. Reactions were initiated by the addition of ATP, the final reaction mixture consisted of 20 μM PIP2, 20 μM ATP, 0.2 Ci [γ-³³P] ATP, 4 nM PI3Kδ. Reactions were incubated for 210 min and terminated by the addition of 40 μL SPA beads suspended in quench buffer: 150 mM potassium phosphate pH 8.0, 20% glycerol. 25 mM EDTA, 400 μM ATP. The final concentration of SPA beads was 1.0 mg/mL. After the plate sealing, plates were shaken overnight at room temperature and centrifuged at 1800 rpm for 10 minutes, the radioactivity of the product was determined by scintillation counting on Topcount (Perkin-Elmer). IC₅₀ determination was performed by fitting the curve of percent control activity versus the log of the inhibitor concentration using the GraphPad Prism 3.0 software.

(R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one was found have an IC₅₀ of <10 nM in the assay of Example 8.

### Example 9: B Cell Proliferation Assay

To acquire B cells, human PBMC are isolated from the peripheral blood of normal, drug free donors by standard density gradient centrifugation on Ficoll-Hypague (GE Healthcare, Piscataway, N.J.) and incubated with anti-CD19 microbeads (Miltenyi Biotech, Auburn, Calif.). The B cells are then purified by positive immunosorting using an autoMacs (Miltenyi Biotech) according to the manufacture's instruction.

The purified B cells (2×10⁵/well/200 μL) are cultured in 96-well ultra-low binding plates (Corning, Corning, N.Y.) in RPMI1640, 10% FBS and goat F(ab′)2 anti-human IgM (10 μg/ml) (Invitrogen, Carlsbad, Calif.) in the presence of different amount of test compounds for three days. [³H]-thymidine (1 μCi/well) (PerkinElmer, Boston, Mass.) in PBS is then added to the B cell cultures for an additional 12 hours before the incorporated radioactivity is separated by filtration with water through GF/B filters (Packard Bioscience, Meriden, Conn.) and measured by liquid scintillation counting with a TopCount (Packard Bioscience).

### Example 10: Pfeiffer Cell Proliferation Assay

Pfeiffer cell line (diffuse large B cell lymphoma) are purchased from ATCC (Manassas, Va.) and maintained in the culture medium recommended (RPMI and 10% FBS). To measure the anti-proliferation activity of the compounds, the Pfeiffer cells are plated with the culture medium (2×10³ cells/well/per 200 μl) into 96-well ultra-low binding plates (Corning, Corning, N.Y.), in the presence or absence of a concentration range of test compounds. After 3-4 days, [3H]-thymidine (1 Ci/well) (PerkinElmer, Boston, Mass.) in PBS is then added to the cell culture for an additional 12 hours before the incorporated radioactivity is separated by filtration with water through GF/B filters (Packard Bioscience, Meridenj, Conn.) and measured by liquid scintillation counting with a TopCount (Packard Bioscience).

### Example 11: Akt Phosphorylation Assay

Ramos cells (B lymphocyte from Burkitts lymphoma) are obtained from ATCC (Manassas, Va.) and maintained in RPMI1640 and 10% FBS. The cells (3×10⁷ cells/tube/3 mL in RPMI) are incubated with different amounts of test compounds for 2 hrs at 37° C. and then stimulated with goat F(ab′)2 anti-human IgM (5 μg/mL) (Invitrogen) for 17 minutes in a 37° C. water bath. The stimulated cells are spun down at 4° C. with centrifugation and whole cell extracts are prepared using 300 μL lysis buffer (Cell Signaling Technology, Danvers, Mass.). The resulting lysates are sonicated and supernatants are collected. The phosphorylation level of Akt in the supernatants are analyzed by using PathScan phospho-Akt1 (Ser473) sandwich ELISA kits (Cell Signaling Technology) according to the manufacturer's instruction.

### Example 12. Permeability and Eye Irritation Studies of the Topical Formulations

An in vitro eye irritation study was conducted to determine if a representative topical formulation was an eye irritant. A topical formulation of 0.1% w/w of Compound A HCl was prepared and is shown in Table 4 below. The formulation was tested in a bovine corneal opacity and permeability (BCOP) test. The results are shown in Table 5 below. The 0.1% w/w formulation resulted in a designation of “No Category”. This means it is not classified as an eye irritant. It causes no serious eye damage according to the globally harmonized system of classification and labeling of chemicals.

TABLE 4
Topical Formulation

| | 0.1% |
|---|---|
| | % w/w |
| Compound A HCl* | 0.108 |
| Purified Water, USP | qs |
| Disodium EDTA, USP | 0.05 |
| PEG 300, NF | 7 |
| Propylene Glycol, USP | 10 |
| Xanthan Gum, NF | 0.3 |
| White Petrolatum, USP | 7 |
| Light Mineral Oil, NF | 4 |
| Glyceryl mono and distearate, NF | 3 |
| Cetyl Alcohol, NF | 3 |
| Stearyl Alcohol, NF | 1.75 |
| Med. Chain Triglycerides, NF | 6 |
| Polysorbate 20, NF | 1.25 |
| Phenoxyethanol, NF | 0.5 |
| Total | 100 |

*0.108% of Compound A HCl is equivalent to 0.1% of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one free base.

TABLE 5
BCOP score with negative and positive controls

| | Mean Opacity | Mean Permeability | Mean In vitro |
|---|---|---|---|
| Treatment | (lux) | (AU) | Irritancy Score |
| Negative control | 0.2 | −0.046 | −0.5 |
| Positive control | 20.1 | 0.580 | 28.8 |
| Compound A 0.1% | 2.1 | −0.001 | 2.0 |
| w/w formulation | | | |

### Example 13. Compound a Concentration in Topical Formulation

The concentration of Compound A HCl was also investigated on a rodent model. Formulations with various strengths between 0.5-2% of Compound A HCl were applied to a mouse inflammatory model. Table 6 below lists the formulations tested.

TABLE 6
Topical Formulations

| | 0% | 0.5% | 1% | 2% |
|---|---|---|---|---|
| | % w/w | % w/w | % w/w | % w/w |
| Compound A HCl* | 0 | 0.5 | 1.0 | 2.0 |
| Purified Water, USP | qs | qs | qs | qs |
| Disodium EDTA, USP | 0.05 | 0.05 | 0.05 | 0.05 |
| PEG 300, NF | 0 | 0 | 0 | 0 |
| Propylene Glycol, USP | 15 | 15 | 15 | 15 |
| Xanthan Gum, NF | 0.4 | 0.4 | 0.4 | 0.4 |
| White Petrolatum, USP | 7 | 7 | 7 | 7 |
| Light Mineral Oil, NF | 4 | 4 | 4 | 4 |
| Glyceryl mono and | 3 | 3 | 3 | 3 |
| distearate, NF | | | | |
| Cetyl Alcohol, NF | 3 | 3 | 3 | 3 |
| Stearyl Alcohol, NF | 1.75 | 1.75 | 1.75 | 1.75 |
| Med. Chain Triglycerides, | 5 | 5 | 5 | 5 |
| NF | | | | |
| Polysorbate 20, NF | 1.25 | 1.25 | 1.25 | 1.25 |
| Cyclopentasiloxan | 1 | 1 | 1 | 1 |
| Methylparaben | 0.1 | 0.1 | 0.1 | 0.1 |
| Propylparaben | 0.05 | 0.05 | 0.05 | 0.05 |
| PEG 200, NF | 7 | 7 | 7 | 7 |
| Phenoxyethanol, NF | 0.5 | 0.5 | 0.5 | 0.5 |
| Total | 100 | 100 | 100 | 100 |

*1.08% of Compound A HCl is equivalent to 1% of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one free base.

These formulations were investigated on a CXCL13 B cell mouse model. CXCL13 was injected intradermally to one mouse ear on Day 0, 2, 4, and 7. Potential topical formulations were topically applied to the same CXCL13 induced ear of mice, with the counter ear acting as an untreated control. Formulations (20 ug per application) were applied twice per day (B.ID.) for eight days. Ears were measured by engineer's calipers for swelling.

Results of the study indicated an inflammatory response for all the formulations, including the placebo vehicle formulation. As such, it is believed that an excipient may be contributing to the cause of the inflammatory response in the animal model. For example, PEG200 may be the cause of the inflammatory response.

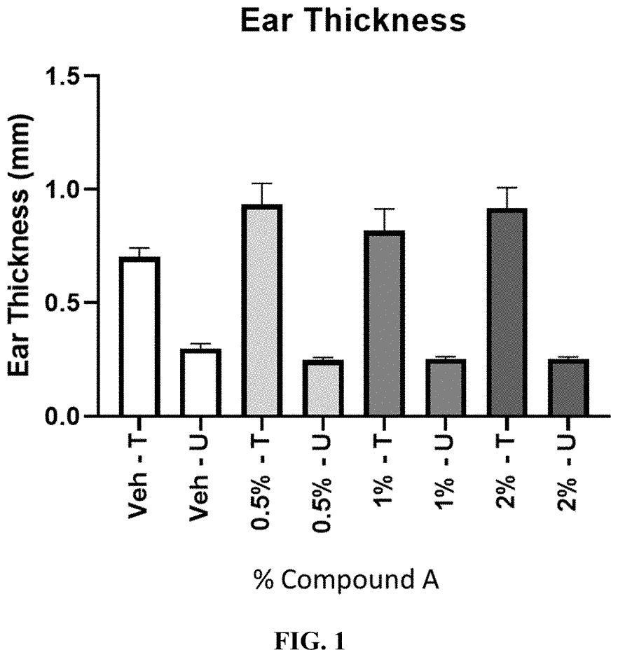

Ear Swelling measured of the treated ear at the end of the study showed thickness was >2× greater to untreated ear for all treatment groups, including vehicle treated group as shown in FIG. 1. No significant difference of the swelling increase was observed between the treatment groups, suggesting an excipient in the formulation is contributing to the cause of inflammatory response in this animal model.

### Example 14. Testing PEG200, PEG 400, and Propylene Glycol in the Topical Formulation

The inflammatory response of PEG200, PEG400, and high amounts of PG in the topical formulations was investigated. Table 7 below lists the formulations tested. None of the formulations contain Compound A.

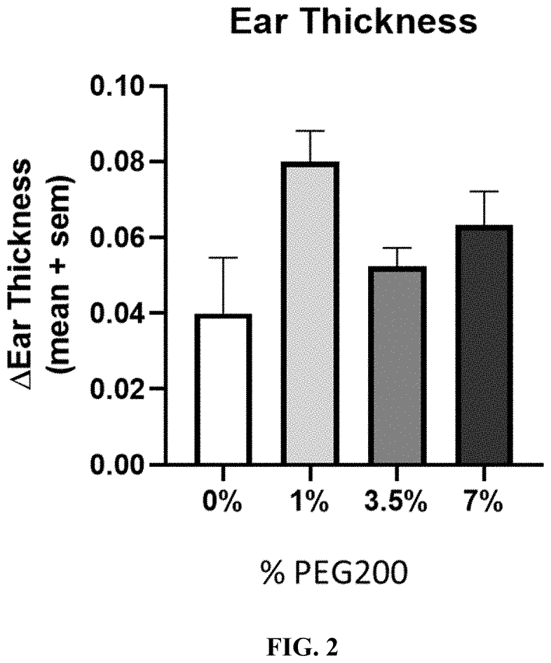

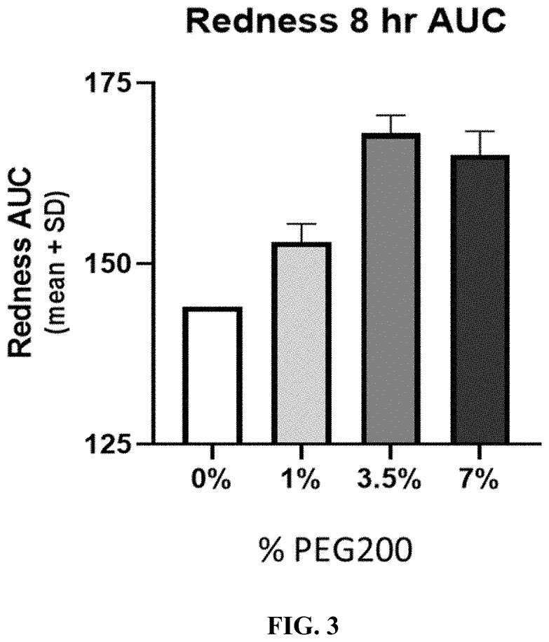

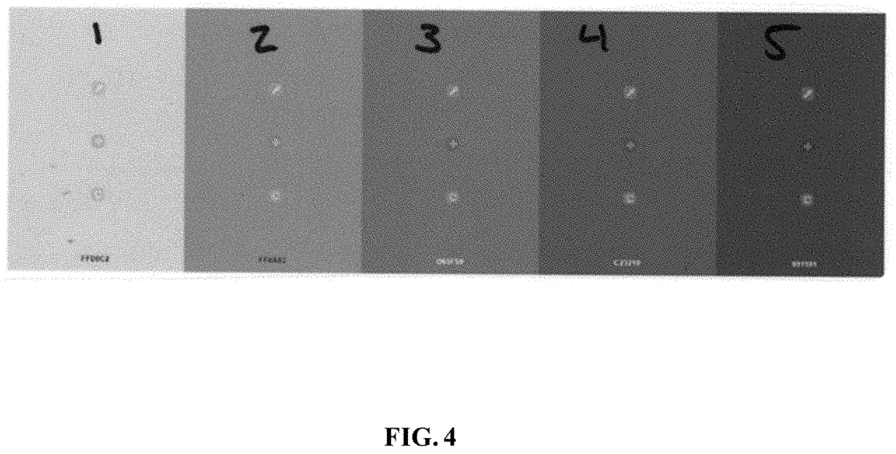

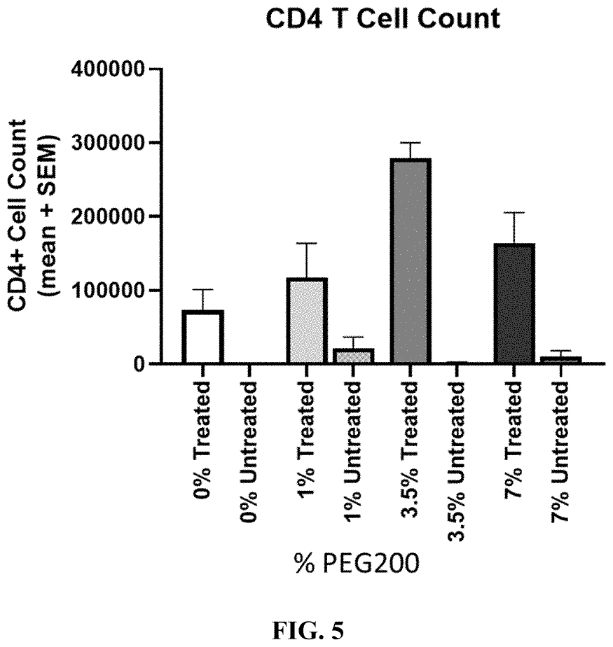

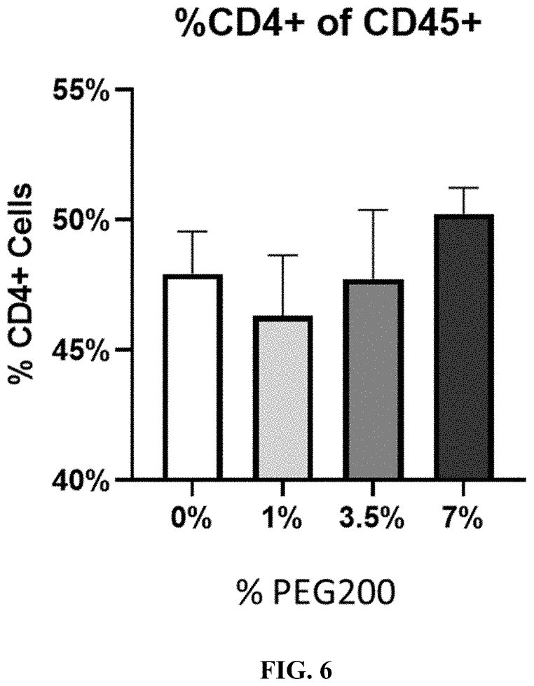

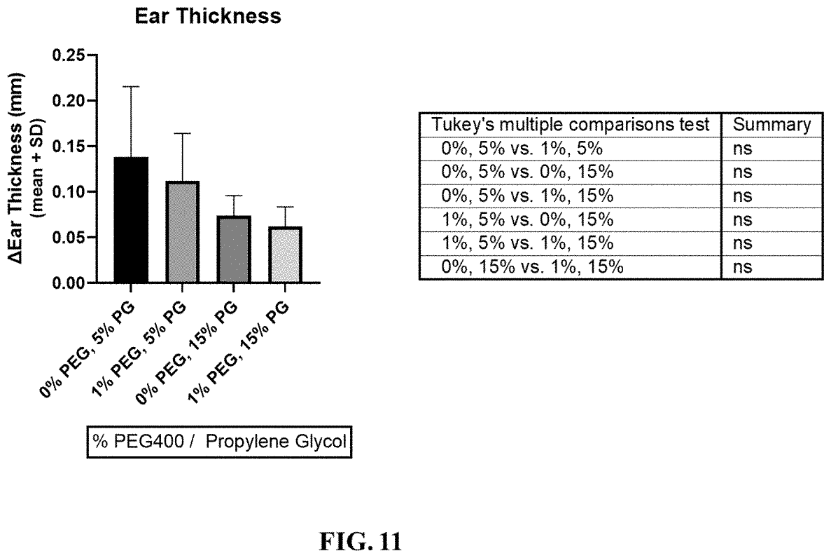

These formulations were investigated on a mouse model. Potential topical formulations were topically applied to one ear of healthy, normal, mice with the counter ear acting as an untreated control. Formulations (20 ug per application) were applied twice per day (B.ID.) for five days. Ears were visually observed for signs of redness (FIG. 3) and measured by engineer's calipers for swelling (FIG. 2). A custom redness scale was created for the redness observations (FIG. 4). The beginning of the scale (1) is roughly equivalent to a healthy, untreated Balb/C mouse ear and increases in redness with increasing values. Redness scale 5 represents an exceptionally red Balb/C mouse ear. The mouse ears were compared visually to the custom redness scale and given a score. At the end of the experiment tissues were collected and immune system activation quantified by flow cytometry (FIGS. 5 and 6 measuring CD4+ T cell count and CD45+ lymphocyte frequency, respectively). The lower concentrations of PEG200 were associated to a reduction in an inflammation response. The study indicated that a lower level of inflammation was associated with decreasing concentrations of PEG200. The PG concentration was held at 1500 w/w. This level of PG did not significantly result in an inflammatory response as seen in the formulation with low levels of PEG200. The data also indicated that the concentrations of PEG400 at 1% and propylene glycol at 500 to 15% did not show a statistical difference in inflammation (FIG. 11). The non-inflammatory properties of PEG400 compared with PEG200 allowed for the investigation of the anti-inflammatory properties of Compound A, i.e., in Example 18.

TABLE 7
Topical Formulations

| | % w/w | % w/w | % w/w | % w/w | % w/w | % w/w | % w/w | % w/w |
|---|---|---|---|---|---|---|---|---|
| Purified Water, USP | qs | qs | qs | qs | qs | qs | qs | qs |
| Disodium EDTA, USP | 0.05 | 0.05 | 0.05 | 0.05 | 0.05 | 0.05 | 0.05 | 0.05 |
| Propylene Glycol, USP | 15 | 15 | 15 | 15 | 15 | 15 | 5 | 5 |
| Xanthan Gum, NF | 0.4 | 0.4 | 0.4 | 0.4 | 0.4 | 0.4 | 0.4 | 0.4 |
| White Petrolatum, USP | 7 | 7 | 7 | 7 | 7 | 7 | 7 | 7 |
| Light Mineral Oil, NF | 4 | 4 | 4 | 4 | 4 | 4 | 4 | 4 |
| Glyceryl mono and | 3 | 3 | 3 | 3 | 3 | 3 | 3 | 3 |
| distearate, NF | | | | | | | | |
| Cetyl Alcohol, NF | 3 | 3 | 3 | 3 | 3 | 3 | 3 | 3 |
| Stearyl Alcohol, NF | 1.75 | 1.75 | 1.75 | 1.75 | 1.75 | 1.75 | 1.75 | 1.75 |
| Med. Chain | 5 | 5 | 5 | 5 | 5 | 5 | 5 | 5 |
| Triglycerides, NF | | | | | | | | |
| Polysorbate 20, NF | 1.25 | 1.25 | 1.25 | 1.25 | 1.25 | 1.25 | 1.25 | 1.25 |
| Methylparaben | 0.1 | 0.1 | 0.1 | 0.1 | 0.1 | 0.1 | 0.1 | 0.1 |
| Propylparaben | 0.05 | 0.05 | 0.05 | 0.05 | 0.05 | 0.05 | 0.05 | 0.05 |
| PEG 200, NF | 0 | 1 | 3.5 | 7 | 0 | 0 | 0 | 0 |
| PEG 400 | 0 | 0 | 0 | 0 | 1 | 0 | 1 | 0 |
| Phenoxyethanol, NF | 0.5 | 0.5 | 0.5 | 0.5 | 0.5 | 0.5 | 0.5 | 0.5 |
| Total | 100 | 100 | 100 | 100 | | | | |

### Example 15. Phase Separation Stability Testing

The physical stability of the topical formulations was tested. Accelerated stability studies were performed to examine if the oil and aqueous phase of particular formulations are homogenous. A homogenous topical formulation is conducive for the delivery of active pharmaceutical ingredients. Under accelerated conditions visual and microscopic observations were made to assess the separation of the two main formulation phases (aqueous and oil phases). Testing conditions were at room temperature (25° C./60% relative humidity) and at accelerated condition (40° C./70%0 relative humidity). Tables 8-10 below lists the formulations tested.

The formulation with the label of 0.1% and the prototypes 14 and 18 (i.e., PT14 and PT18 in Table 10) were physically stable formulations. Physical separation, indicating instability, was observed for the other formulations listed in tables. Physical instability was evaluated by visual observations and microscopic imaging. This test indicates that a stable formulation requires a concentration of xanthan gum of 0.2% w/w or higher, if % included, and a concentration of % PEG400 of 5 w/w or higher, if included. Phase separation also resulted from the lower concentrations of propylene glycol (Tables 8 and 9). Elimination of dimethicone did not cause instability (Table 8). Phase separation was observed in the absence of white petrolatum in comparison with the formulations containing white petrolatum (Table 10).

TABLE 8
Topical Formulations PT2-PT5

| | 0.1% | PT2 | PT3 | PT4 | PT5 |
|---|---|---|---|---|---|
| | % w/w | % w/w | % w/w | % w/w | % w/w |
| Compound A | 0.1 | 0.1 | 0.1 | 0.1 | 0.1 |
| Purified Water, USP | qs | qs | qs | qs | qs |
| Disodium EDTA, | 0.05 | 0.05 | 0.05 | 0.05 | 0.05 |
| USP | | | | | |
| Propylene Glycol, | 10 | 5 | 2.5 | 1 | 0 |
| USP | | | | | |
| Xanthan Gum, NF | 0.4 | 0 | 0 | 0 | 0 |
| White Petrolatum, | 7 | 7 | 7 | 7 | 7 |
| USP | | | | | |
| Light Mineral Oil, | 4 | 4 | 4 | 4 | 4 |
| NF | | | | | |
| Glyceryl mono and | 3 | 3 | 3 | 3 | 3 |
| distearate, NF | | | | | |
| Cetyl Alcohol, NF | 3 | 3 | 3 | 3 | 3 |
| Stearyl Alcohol, NF | 1.75 | 1.75 | 1.75 | 1.75 | 1.75 |
| Med. Chain | 5 | 5 | 5 | 5 | 5 |
| Triglycerides, NF | | | | | |
| Polysorbate 20, NF | 1.25 | 1.25 | 1.25 | 1.25 | 1.25 |
| Methylparaben | 0.1 | 0 | 0 | 0 | 0 |
| Propylparaben | 0.05 | 0 | 0 | 0 | 0 |
| PEG 400 | 5 | 5 | 5 | 5 | 5 |
| Benzyl Alcohol | 0 | 1 | 1 | 1 | 1 |
| Phenoxyethanol, NF | 0.5 | 0.5 | 0.5 | 0.5 | 0.5 |
| Total | 100 | 100 | 100 | 100 | 100 |
| % LC (4 weeks) | 99.4% | 109.1% | ND | ND | ND |
| 25° C./60% RH | 107.6% | 107.6% | | | |
| 40° C./70% RH | | | | | |
| Phase separation | None | None | Yes | Yes | Yes |

*ND—due to phase separation
LC—label claim

TABLE 9
Topical Formulations PT6-PT13

| | PT6 | PT7 | PT8 | PT9 | PT10 | PT11 | PT12 | PT13 |
|---|---|---|---|---|---|---|---|---|
| | % w/w | % w/w | % w/w | % w/w | % w/w | % w/w | % w/w | % w/w |
| Compound A | 0.1 | 0.1 | 0.1 | 0.1 | 0.1 | 0.1 | 0.1 | 0.1 |
| Purified Water, | qs | qs | qs | qs | qs | qs | qs | qs |
| USP | | | | | | | | |
| Disodium EDTA, | 0.05 | 0.05 | 0.05 | 0.05 | 0.05 | 0.05 | 0.05 | 0.05 |
| USP | | | | | | | | |
| Propylene Glycol, | 0 | 5 | 2.5 | 1 | 5 | 2.5 | 1 | 0 |
| USP | | | | | | | | |
| White Petrolatum, | 7 | 7 | 7 | 7 | 7 | 7 | 7 | 7 |
| USP | | | | | | | | |
| Light Mineral Oil, | 4 | 4 | 4 | 4 | 4 | 4 | 4 | 4 |
| NF | | | | | | | | |
| Glyceryl mono and | 3 | 3 | 3 | 3 | 3 | 3 | 3 | 3 |
| distearate, NF | | | | | | | | |
| Cetyl Alcohol, NF | 3 | 3 | 3 | 3 | 3 | 3 | 3 | 3 |
| Stearyl Alcohol, | 1.75 | 1.75 | 1.75 | 1.75 | 1.75 | 1.75 | 1.75 | 1.75 |
| NF | | | | | | | | |
| Med. Chain | 5 | 5 | 5 | 5 | 5 | 5 | 5 | 5 |
| Triglycerides, NF | | | | | | | | |
| Polysorbate 20, NF | 1.25 | 1.25 | 1.25 | 1.25 | 1.25 | 1.25 | 1.25 | 1.25 |
| PEG 400 | 5 | 2.5 | 2.5 | 2.5 | 0 | 0 | 0 | 0 |
| Dimethicone | 0 | 0 | 0 | 0 | 1 | 1 | 1 | 1 |
| Benzyl Alcohol | 1 | 1 | 1 | 1 | 1 | 1 | 1 | 1 |
| Phenoxyethanol, NF | 0.5 | 0.5 | 0.5 | 0.5 | 0.5 | 0.5 | 0.5 | 0.5 |
| Total | 100 | 100 | 100 | 100 | 100 | 100 | 100 | 100 |
| Phase separation | Yes | No | No | Yes | No | Yes | Yes | Yes |

In some formulations, xanthan gum was incorporated for the stabilization of the microstructure and viscosity. The stability of the formulations were evaluated by visual observations, microscopic imaging, and TIPLC analysis of the percentage label claim.

For PT 16 and PT 17, these formulations showed a distinct separation of the aqueous phase and microscopic imaging indicated the formulations were not homogenous. Microscopic analysis indicate the formulation losing a homogenous consistency, indicating a disruption in the microstructure.

TABLE 10
Topical Formulations PT14-PT19

| | PT14 | PT15 | PT16 | PT17 | PT18 | PT19 |
|---|---|---|---|---|---|---|
| | % w/w | % w/w | % w/w | % w/w | % w/w | % w/w |
| Compound A | 0.1 | 0.1 | 0.1 | 0.1 | 0.1 | 0.1 |
| Purified Water, | qs | qs | qs | qs | qs | qs |
| USP | | | | | | |
| Disodium EDTA, | 0.05 | 0.05 | 0.05 | 0.05 | 0.05 | 0.05 |
| USP | | | | | | |
| Propylene Glycol, | 5 | 5 | 5 | 5 | 5 | 5 |
| USP | | | | | | |
| Xanthan Gum | 0.2 | 0.1 | 0.2 | 0.1 | 0.2 | 0.1 |
| White Petrolatum, | 7 | 7 | 0 | 0 | 7 | 7 |
| USP | | | | | | |
| Light Mineral Oil, | 4 | 4 | 4 | 4 | 0 | 0 |
| NF | | | | | | |
| Glyceryl mono | 3 | 3 | 3 | 3 | 3 | 3 |
| and distearate, NF | | | | | | |
| Cetyl Alcohol, NF | 3 | 3 | 3 | 3 | 3 | 3 |
| Stearyl Alcohol, | 1.75 | 1.75 | 1.75 | 1.75 | 1.75 | 1.75 |
| NF | | | | | | |
| Med. Chain | 5 | 5 | 5 | 5 | 5 | 5 |
| Triglycerides, NF | | | | | | |
| Polysorbate 20, | 1.25 | 1.25 | 1.25 | 1.25 | 1.25 | 1.25 |
| NF | | | | | | |
| PEG 400 | 5 | 5 | 5 | 5 | 5 | 5 |
| Phenoxyethanol, NF | 0.5 | 0.5 | 0.5 | 0.5 | 0.5 | 0.5 |
| Total | 100 | 100 | 100 | 100 | 100 | 100 |
| % LC* | | | | | | |
| 25° C./60% RH | 102.1% | 126.2% | 84.7% | 118.6% | 101.2% | 103.7% |
| 40° C./70% RH | 107.3% | 121.0% | 134.2% | 126.4% | 94.5% | 116.6% |
| Phase separation | No | No | Yes | Yes | No | No |

LC—label claim
*% LC higher than 110% may be attributed to evaporation due to poor sealing of the container during testing.

### Example 16. Additional Alternate Topical Formulations

Gel/hydrogel formulations of Compound A can also be prepared. The Compound A content in the gel/hydrogel formulations can be between 0.01% to 1%. Table 11 below provides some exemplary formulations.

TABLE 11
Gel Topical Formulations

| | | % w/w | % w/w |
|---|---|---|---|
| Compound A | | 0.01-1 | 0.01-1 |
| Purified Water, USP | solvent | qs | qs |
| Propylene Glycol, USP | solvent | 10 | 10 |
| Ethanol | solvent | 10 | 10 |
| Glycerin | humectant | 5 | 5 |
| Poloxamer 188, 338 or | viscosity mod. | 18 | 20 |
| 407 | | | |
| Polysorbate 20, NF | surfactant | 0 | 1 |
| PEG 400 | solvent | 0 | 5 |
| Phenoxyethanol, NF | preservative | 0.5 | 0.5 |
| Total | | 100 | 100 |

Additional alternative topical formulations were prepared as shown in Table 12.

TABLE 12
Additional Alternative Topical
Formulations

| | % w/w | % w/w | % w/w |
|---|---|---|---|
| Compound A | 0.1-1 | 0.1-1 | 0.1-1 |
| Purified Water, | qs | qs | qs |
| USP | | | |
| Disodium EDTA, | 0.05 | 0.05 | 0.05 |
| USP | | | |
| Propylene Glycol, | 10 | 5 | 10 |
| USP | | | |
| Xanthan Gum | 0.4 | 0.3 | 0.3 |
| White Petrolatum, | 7 | 7 | 7 |
| USP | | | |
| Light Mineral Oil, | 4 | 4 | 4 |
| NF | | | |
| Glyceryl mono and | 3 | 3 | 3 |
| distearate, NF | | | |
| Cetyl Alcohol, NF | 3 | 3 | 3 |
| Stearyl Alcohol, | 1.75 | 1.75 | 1.75 |
| NF | | | |
| Methylparaben | 0.1 | 0 | 0 |
| Propylparaben | 0.05 | 0 | 0 |
| Med. Chain | 5 | 5 | 6 |
| Triglycerides, NF | | | |
| Polysorbate 20, NF | 1.25 | 1.25 | 1.25 |
| PEG 300 | 0 | 0 | 7 |
| PEG 400 | 5 | 5 | 0 |
| Phenoxyethanol, | 0.5 | 0.5 | 1.0 |
| NF | | | |
| Total | 100 | 100 | 100 |

Aerosolized foam, non-aerosolized foam, film forming spray and ointment formulations can be prepared as shown in Tables 13-16.

TABLE 13
Aerosolized Foam Formulations

| | | % w/w | % w/w | % w/w | % w/w | % w/w |
|---|---|---|---|---|---|---|
| Compound A | API | 0.01-1 | 0.01-1 | 0.01-1 | 0.01-1 | 0.01-1 |
| Purified Water, USP | solvent | qs | qs | qs | qs | qs |
| Citric Acid | Antioxidant | 0.1 | 0.1 | 0.1 | 0.1 | 0.1 |
| Sodium Citrate | Buffering agent | 0.3 | 0.3 | 0.3 | 0.3 | 0.3 |
| Dihydrate | | | | | | |
| Oleth 10 | Emulsifier | 8 | 0 | 0 | 0 | 0 |
| | surfactant | | | | | |
| PEG 40 stearate | Emulsifier/Solvent | 0 | 3 | 0 | 0 | 0 |
| Disodium EDTA | Chelating agent | 0.1 | 0.1 | 0.1 | 0.1 | 0.1 |
| Polysorbate 80 | Surfactant | 0 | 0 | 7 | 0 | 0 |
| Polysorbate 20 | Surfactant | 0 | 0 | 0 | 3 | 0 |
| Ceteareth-20 | Surfactant | 0 | 0 | 0 | 0 | 3 |
| Cetyl alcohol | Binding Agent | 1.5 | 1.5 | 1.5 | 1.5 | 1.5 |
| PEG 300 | Solvent | 4 | 4 | 0 | 0 | 0 |
| PEG 400 | Solvent | 0 | 0 | 4 | 4 | 0 |
| Glycerin | Humectant | 0 | 0 | 0 | 0 | 10 |
| BHT | Antioxidant | 0.1 | 0.1 | 0.1 | 0.1 | 0.1 |
| Benzyl Alcohol | Preservative | 0.5 | 0.5 | 0.5 | 0.5 | 0.5 |
| Emulsifying Wax | Emulsifier | 5 | 0 | 0 | 0 | 0 |
| Glyceryl stearate | Surfactant/stabilizer | 0 | 2.5 | 0 | 0 | 0 |
| Petrolatum | Emollient | 0 | 0 | 1 | 1 | 20 |
| Mineral Oil | Emollient | 0 | 0 | 1 | 1 | 20 |
| Natosol 250 H | Thickener | 0 | 0.3 | 0 | 0 | 0 |
| Propylene Glycol | Solvent | 10 | 10 | 10 | 10 | 10 |
| Carbopol 981 | Emulsifier | 0 | 0 | 0 | 0.1 | 0 |
| Castor Oil | Emollient | 0 | 0 | 0 | 0.3 | 0 |
| Propellant | | qs | qs | qs | qs | qs |
| Total | | 100 | 100 | 100 | 100 | 100 |

TABLE 14
Non Aerosolized Foam Formulation

| | | % w/w |
|---|---|---|
| Compound A | | 0.01-1 |
| Purified Water, USP | solvent | qs |
| Pol oxamer 188 | emulsifier | 3 |
| BHT | preservative | 0.1 |
| Total | | 100 |

TABLE 15
Film Forming Spray

| | | % w/w |
|---|---|---|
| Compound A | | 0.01-1 |
| Purified Water, USP | solvent | qs |
| Film Former | | 3.0 |
| Ethanol | solvent | 85 |
| Plasticizer | | 1.0 |
| Phenoxyethanol | preservative | 1.0 |
| Total | | 100 |

TABLE 16
Ointment

| | | % w/w |
|---|---|---|
| Compound A | | 0.01-1 |
| Castor Oil Hydrogenated | Emollient | 2.0 |
| Glyceryl Monostearate | Emollient | 4.0 |
| Cocoyl Caprylocaprate | Emollient/solvent | 19.0 |
| Octyldodecanol | Emollient/solvent | 19.0 |
| Emulsifying Wax | Viscosity modifier | 10.0 |
| Cetyl Palmitate | Emollient/solvent | 7.0 |
| Isopropyl Myristate | Emollient/solvent | 10.0 |
| Medium Chain Triglycerides | Emollient | 9.0 |

### Example 17. Clinical Trial

A Phase 1, double-blind, randomized, placebo controlled multiple-dose study to assess the safety, tolerability and pharmacokinetics of Compound A formulations applied topically to healthy adult participants is conducted. The objectives of the study are to determine the safety and tolerability of a cream formulation of Compound A or placebo when applied QD and BID to healthy participants, and to evaluate systemic pharmacokinetic parameters. PK parameters will be monitored, including Cₘₐₓ, Tₘₐₓ, AUC₀₋ₜ, and AUC_{0-∞}.

In each of the 3 Cohorts, 9 participants will apply Compound A cream and 3 participants will apply placebo cream. Each participant in Cohorts 1 through 3 will receive either multiple doses of Compound A or placebo, dosed QD or BID, during the study. Participants will have 10% or 20% BSA coverage for application of the Compound A cream or placebo. The estimated duration of study is up to 28 days for screening, 10 days from check-in for treatment, and approximately 14 days±3 days from the last dose of study drug through safety follow-up (i.e., study day 24±3 days). It is estimated that an individual will participate for approximately 2 to 2.5 months.

Cohort 1 will involve Compound A 0.01% cream or placebo applied QD to 10% BSA for 10 days. Cohort 2 will involve Compound A 0.01% cream or placebo applied BID to 10% BSA for 10 days. Cohort 3 will involve Compound A 0.01% cream or placebo applied QD to 20% BSA for 10 days. The application area for 10% BSA will be one entire arm and hand, front and back and the anterior neck. The application area for 20% BSA will be both entire arms and hands, front and back, and both anterior and posterior neck. The formulations used for the study are listed in Table 17 below.

TABLE 17
Topical Formulation

| | Placebo | 0.01% |
|---|---|---|
| | % w/w | % w/w |
| Compound A HCl* | 0 | 0.0108 |
| Purified Water, USP | qs | qs |
| Disodium EDTA, USP | 0.05 | 0.05 |
| PEG 300, NF | 7 | 7 |
| Propylene Glycol, USP | 10 | 10 |
| Xanthan Gum, NF | 0.35 | 0.35 |
| White Petrolatum, USP | 7 | 7 |
| Light Mineral Oil, NF | 4 | 4 |
| Glyceryl mono and distearate, NF | 3 | 3 |
| Cetyl Alcohol, NF | 3 | 3 |
| Stearyl Alcohol, NF | 1.75 | 1.75 |
| Med. Chain Triglycerides, NF | 6 | 6 |
| Polysorbate 20, NF | 1.25 | 1.25 |
| Phenoxyethanol, NF | 0.5 | 0.5 |
| Total | 100 | 100 |

*The amount of Compound A HCl is equivalent to the % w/w of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one free base, e.g., 0.0108% of HCl is equivalent to 0.01% free base.

Blood samples for determination of plasma concentrations of Compound A will be collected on Day 1 at pre-application, 1, 2, 4, 8, 12, 24 hours post dose and on Day 10 at pre-application, 1, 2, 4, 8, 12, 24, 36, 48 and 72 hours post-application. Skin biopsies for skin PK will be collected on Days 1, 10, and 13.

The topical formulation of Compound A at 0.01% w/w is used to topically deliver Compound A locally and directly to the target areas of the skin while significantly reducing circulating plasma concentrations. Based on initial studies it is believed that topical administration of a strength of 0.01% may result in mean plasma concentration of approximately 1 nM at steady state. It is also believed that dermal concentrations of ˜150 nM can be achieved, which would exceed the IC90 value of ˜20 nM in the dermis, with limited systemic exposure.

Inclusion Criteria

Participants are eligible to be included in the study only if all of the following criteria apply:

Is male or female, between 18 and 65 years of age (inclusive) at Screening.

Has a BMI between 18.5 and 35.0 kg/m² (inclusive) at Screening.

Is in good general health, as determined by the Investigator, without clinically significant medical history.

Normal or non-clinically significant findings on a physical examination, 12-lead ECG, and vital signs, as determined by the Investigator.

Clinical laboratory values within the normal limits as defined by the clinical laboratory, unless the Investigator decides that out-of-range values are not clinically significant.

Willingness to avoid pregnancy or fathering children based on the criteria below.

Male participants with childbearing potential must agree to take appropriate precautions to avoid fathering children (with at least 99% certainty) from screening through 90 days, corresponding to time needed to eliminate study drug after the last dose of study drug, and must refrain from donating sperm during this period. Permitted methods that are at least 99% effective in preventing pregnancy should be communicated to the participants and their understanding confirmed.

Female participants with childbearing potential must have a negative SPT at screening and UPT before the first dose at check-in on Day −1 and must agree to take appropriate precautions to avoid pregnancy (with at least 99% certainty) from screening through safety follow-up. Permitted methods that are at least 99% effective in preventing pregnancy should be communicated to the participants and their understanding confirmed.

Female participants without childbearing potential (i.e., surgically sterile with a hysterectomy and/or bilateral oophorectomy OR≥12 months of amenorrhea) are eligible.

Has a negative screen for drugs of abuse, alcohol, HBs-Ag, HBs-Ab, HCV-Ag, HCV-Ab, and HIV at Screening; and negative drugs of abuse and alcohol screen on Day −1.

Is free of any systemic or dermatological disorder, which, in the opinion of the Investigator, will interfere with the study results or increase the risk of AEs.

Is of any skin type or race, providing the skin pigmentation will allow discernment of erythema.

Is willing and able to follow study instructions and likely to complete all study requirements.

Exclusion Criteria

Participants are excluded from the study if any of the following criteria apply:

Is a female who is pregnant, plans to become pregnant during the study, or is breast-feeding a child.

Has an open wound and/or sunburn(s) in the dosing area. Participants who have a wound and/or sunburn at Screening which is anticipated to resolve before Day −1 may be enrolled.

Has any clinically significant central nervous system, cardiac, pulmonary, renal, gastrointestinal, respiratory, metabolic conditions (or history), or other pathological or physiological conditions, including a history of paralytic ileus, glaucoma, prostatic hypertrophy or tachycardia, that might interfere with the study result in the Investigator's opinion.

Has any condition, which in the Investigator's opinion, puts the participant at significant risk, could confound the study results or may interfere significantly with the participant's participation in the study.

Is not willing to refrain from using systemic/topical analgesics such as aspirin (81 mg daily aspirin will be allowed), Aleve, Motrin, Advil, or Nuprin for 72 hours prior to and during the study (occasional use of acetaminophen will be permitted).

Has had an immunization within 10 days of study entry.

Has a history of, or is currently being treated for type I or type II diabetes.

Anticipated need for surgery or hospitalization during the study.

Has consumed alcohol within 48 hours prior to Day 1 or refuses to abstain from alcohol throughout the duration of the study.

Has consumed caffeine (i.e., coffee, tea, caffeinated soda, chocolate) within 48 hours prior to Day 1 or refuses to abstain from caffeine throughout the duration of the study.

Has consumed Seville oranges, grapefruit or grapefruit juice, pomelos, exotic citrus fruits, grapefruit hybrids, or fruit juices containing products for 72 hours prior to Day 1 or refuses to abstain throughout the duration of the study.

Has a history of heavy smoking (i.e., more than 10 cigarettes a day or the tobacco/nicotine equivalent) within 3 months of screening, or refuses to abstain from tobacco or nicotine-containing products throughout the duration of the study.

Has a donation or loss of blood (excluding volume drawn at Screening) of >450 mL within 3 months of Day 1.

Has an active or lifetime infection (e.g., HIV, hepatitis, tuberculosis, syphilis), or a history of severe infection during the 30 days prior to Screening.

Has a history of serious skin disease (as determined by the Investigator), such as skin cancer, psoriasis, eczema, or stasis dermatitis.

Chronic or current active infectious disease requiring systemic antibiotics, antifungal, or antiviral treatment.

Prior treatment with a PI3Kδ inhibitor for any indication.

Has used prescription or over-the-counter medications within 30 days of IP administration, with the exception of contraceptive medications, unless agreed as non-clinically relevant by the Investigator and Sponsor;

Known hypersensitivity or severe reaction to Compound A or excipients of Compound A.

Inability or unlikeliness of the participant to comply with the dose schedule and study evaluations, in the opinion of the investigator.

Any condition that would, in the investigator's judgment, interfere with full participation in the study, including administration of Compound A cream and attending required study visits; pose a significant risk to the participant; or interfere with interpretation of study data.

Inability of the participant (or legally authorized representative) to comprehend the ICF or unwillingness to sign the ICF.

### Example 18. Anti-Inflammatory Studies

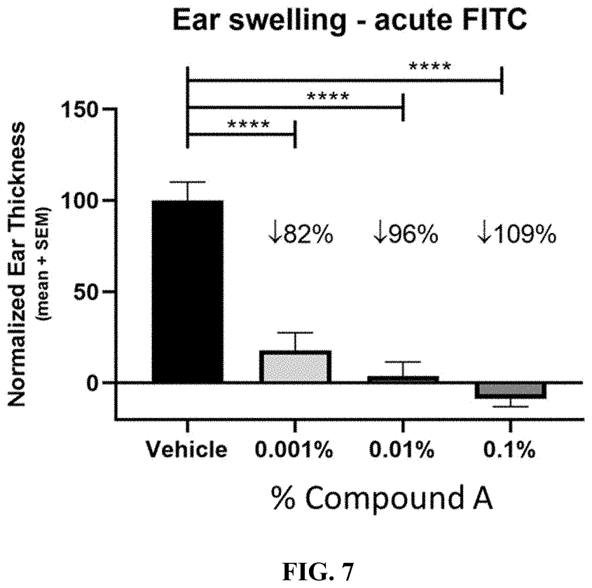

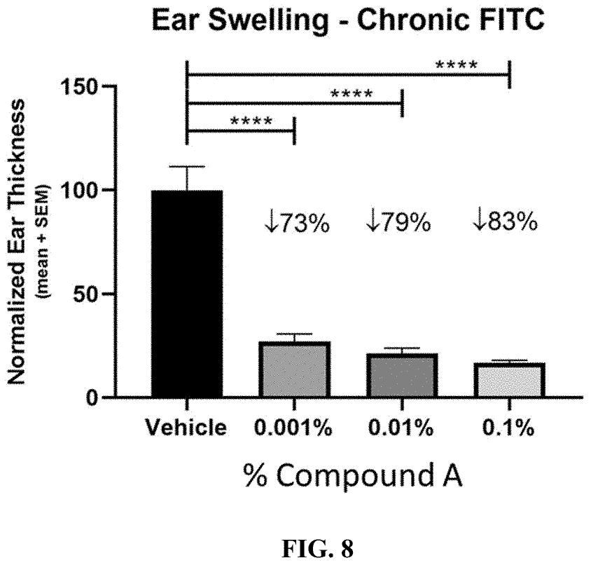

To assess the anti-inflammatory properties of the topical formulations, the formulations were evaluated in an FITC induced dermatitis model (both acute and chronic). Chemically induced dermatitis with small molecule agents, such as FITC, elicit a strong Th2 response in mice, and therefore resemble some aspects of AD. In this model, sensitization to FITC is achieved by initial abdominal challenge and subsequent ear challenge inducing epidermal thickening of challenged ear. The dermatitis phenotype induced by challenges of FITC onto the mouse ear on Day 8 for the acute model and a more sustained, greater thickening from repeat challenge on Days 8, 15, 22, 29, and 36 for the chronic model. Ear swelling was quantified compared to the vehicle treated group at takedown. The topical formulations tested contained from 0.001% to 0.1% w/w of Compound A, i.e., as listed in Table 18 below. They were applied topically twice daily to the ear and significantly inhibited ear swelling at takedown in a dose dependent manner. See FIGS. 7 and 8.

TABLE 18
Topical Formulations

| | 0.001% | 0.01% | 0.1% |
|---|---|---|---|
| | % w/w | % w/w | % w/w |
| Compound A | 0.001 | 0.01 | 0.1 |
| Purified Water, USP | qs | qs | qs |
| Disodium EDTA, USP | 0.05 | 0.05 | 0.05 |
| Propylene Glycol, USP | 10 | 10 | 10 |
| Xanthan Gum, NF | 0.4 | 0.4 | 0.4 |
| White Petrolatum, USP | 7 | 7 | 7 |
| Light Mineral Oil, NF | 4 | 4 | 4 |
| Glyceryl mono and distearate, NF | 3 | 3 | 3 |
| Cetyl Alcohol, NF | 3 | 3 | 3 |
| Stearyl Alcohol, NF | 1.75 | 1.75 | 1.75 |
| Med. Chain Triglycerides, NF | 5 | 5 | 5 |
| Polysorbate 20, NF | 1.25 | 1.25 | 1.25 |
| Methylparaben | 0.1 | 0.1 | 0.1 |
| Propylparaben | 0.05 | 0.05 | 0.05 |
| PEG 400 | 5 | 5 | 5 |
| Benzyl Alcohol | 0 | 0 | 0 |
| Phenoxyethanol, NF | 0.5 | 0.5 | 0.5 |
| Total | 100 | 100 | 100 |

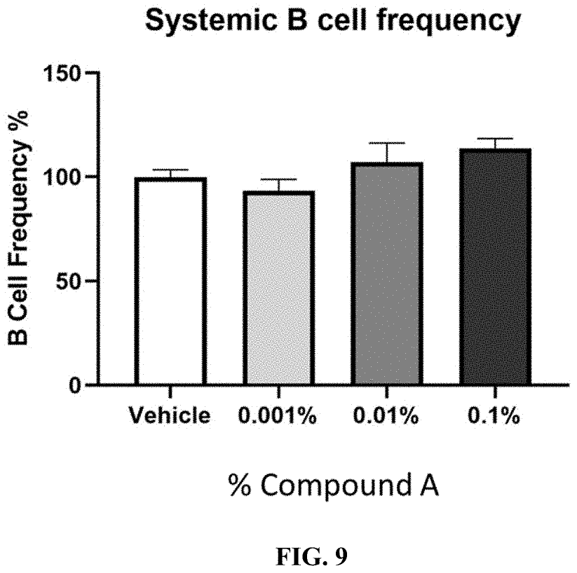

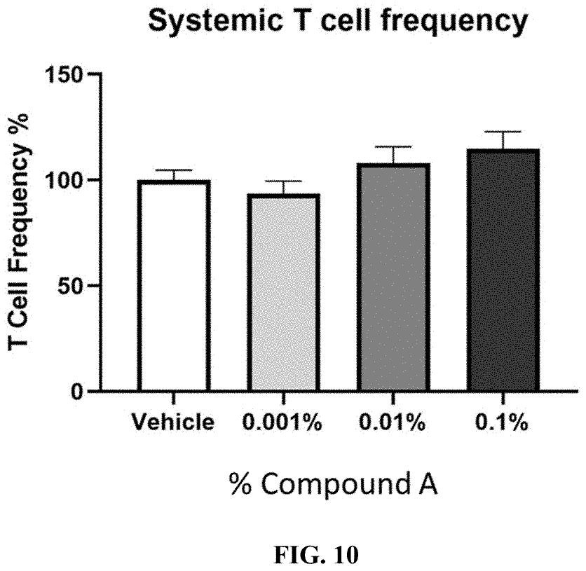

In addition, systemic whole blood CD19⁺ B-cell and CD3⁺ T-cell frequencies were evaluated by flow cytometry in the chronic FITC induced dermatitis model. Treatment with the topical formulations in this chronic dermatitis model did not impact circulating lymphocyte (B and T cells) frequency at any concentration tested (0.001% to 0.1% w/w). See FIGS. 9 and 10. These data suggest a lack of systemic immune suppression at the tested concentrations while achieving a reduction in localized tissue inflammation. It is noted that systemic circulating frequencies of B cells and T cells do not change with topical formulation treatment. Whole blood frequency of CD19+ B cells (FIG. 9) and CD3+ T cells (FIG. 10) are normalized to vehicle group.

### Example 19. In Vitro Permeation Studies

An in vitro permeation test was performed to evaluate topical formulations containing 0.01% w/w and 0.1% w/w of Compound A (Table 19) using ex-vivo human skin dermatomed at 500 to 600 micrometer thickness. This test indicated that 2% of the applied dose (10 microliters) was measured in the dermal layer for the 0.01% and 0.1% w/w topical formulations. The percentage of the dose that penetrated all skin layers over 16 hours ranged from approximately 0.8% to 2% of the applied dose. Skin flux for 0.1% topical formulation was about 4× higher compared with the 0.01% formulation. See Table 20 below.

TABLE 19
Topical Formulations

| | Placebo | 0.01% | 0.1% |
|---|---|---|---|
| | % w/w | % w/w | % w/w |
| Compound A HCl* | 0 | 0.0108 | 0.108 |
| Purified Water, USP | qs | qs | qs |
| Disodium EDTA, USP | 0.05 | 0.05 | 0.05 |
| PEG 300, NF | 7 | 7 | 7 |
| Propylene Glycol, USP | 10 | 10 | 10 |
| Xanthan Gum, NF | 0.35 | 0.40 | 0.30 |
| White Petrolatum, USP | 7 | 7 | 7 |
| Light Mineral Oil, NF | 4 | 4 | 4 |
| Glyceryl mono and distearate, NF | 3 | 3 | 3 |
| Cetyl Alcohol, NF | 3 | 3 | 3 |
| Stearyl Alcohol, NF | 1.75 | 1.75 | 1.75 |
| Med. Chain Triglycerides, NF | 6 | 6 | 6 |
| Polysorbate 20, NF | 1.25 | 1.25 | 1.25 |
| Phenoxyethanol, NF | 0.5 | 0.5 | 0.5 |
| Total | 100 | 100 | 100 |

*The amount of Compound A HCl is equivalent to the % w/w of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one free base, e.g., 0.0108% of HCl is equivalent to 0.01% free base.

TABLE 20
In vitro Permeation Results

| | Topical Formulation | |
|---|---|---|
| | 0.01% w/w | 0.1% w/w |
| Dose | 10 uL | 10 uL |
| Epidermis (ng/cm2) | 117 | 712 |
| Dermis (ng/cm2) | 22 | 178 |
| Cumulative amount through skin layer (ng) | 19 | 78 |
| Skin flux (ng/cm2/h) | 1.2 | 4.9 |

### Example 20. Toxicokinetic Studies

The distribution of Compound A in skin and plasma was tested. A toxicokinetic study was conducted in minipigs, which were dosed with topical formulations at a dose of 0.1% w/w, 1.0% w/w unmodified, and 1.0% w/w modified formulations, applied at 10 mg/cm². The formulations are shown in Table 21 below. The 1% w/w topical formulation was modified by changing the ratio of excipients to facilitate scaling up the topical formulation.

TABLE 21
Topical Formulation

| | % w/w | |
|---|---|---|
| | Unmodified | Modified |
| Compound A | 0.1 or 1 | 1 |
| Purified Water, USP | qs | qs |
| Disodium EDTA, USP | 0.05 | 0.05 |
| PEG300 | 5 | 0 |
| PEG400 | 0 | 7 |
| Propylene Glycol, USP | 5 | 10 |
| Xanthan Gum, NF | 0.3 | 0.3 |
| White Petrolatum, USP | 7 | 7 |
| Light Mineral Oil, NF | 4 | 4 |
| Glyceryl mono and distearate, NF | 3 | 3 |
| Cetyl Alcohol, NF | 3 | 3 |
| Stearyl Alcohol, NF | 1.75 | 1.75 |
| Med. Chain Triglycerides, NF | 5 | 6 |
| Polysorbate 20, NF | 1.25 | 1.25 |
| Phenoxyethanol, NF | 0.5 | 0.5 |
| Total | 100 | 100 |

The concentration of Compound A was measured in the epidermis, dermis, and plasma 24 hours post application. Dermal concentrations in minipigs dosed with 1.0% w/w topical formulation were 8× higher compared with dermal concentrations in minipigs dosed with the 0.1% topical formulation. The epidermal concentration for 1.0% w/w topical formulation was 6× higher compared with the 0.1% w/w topical dose. The epidermal concentrations accounted for the combined drug levels in the stratum corneum and viable epidermis layers (i.e., skin samples were not tape stripped). Skin concentrations and plasma concentration analyses are shown in Table 22 below. Relatedly, the free fraction of Compound A in plasma and homogenized human skin was evaluated by equilibrium dialysis studies. The mean free fraction in human plasma and skin was 7.400 and 3300 respectively. In minipigs, the free fraction in plasma was measured at 3.1%.

TABLE 22
Topical Formulation Toxicokinetic Results

| | | Topical Formulation | | |
|---|---|---|---|---|
| | | 0.1%, | 1.0%, | 1.0%, |
| Tissue | Parameter | unmodified | unmodified | modified |
| Epidermis | nM at 24 h post | 138,000 | 576,000 | 291,000 |
| Dermis | nM at 24 h post | 1,640 | 13,600 | 16,200 |
| Plasma | Cₘₐₓ (nM) | 9.8 | 64.7 | 92.3 |
| | Cₛₛ (nM) | 4.5 | 34.3 | 71.0 |
| | AUC₀₋₂₄ (nM h) | 109 | 823 | 1703 |

### Example 21. Testing Xanthan Gum in the Topical Formulation

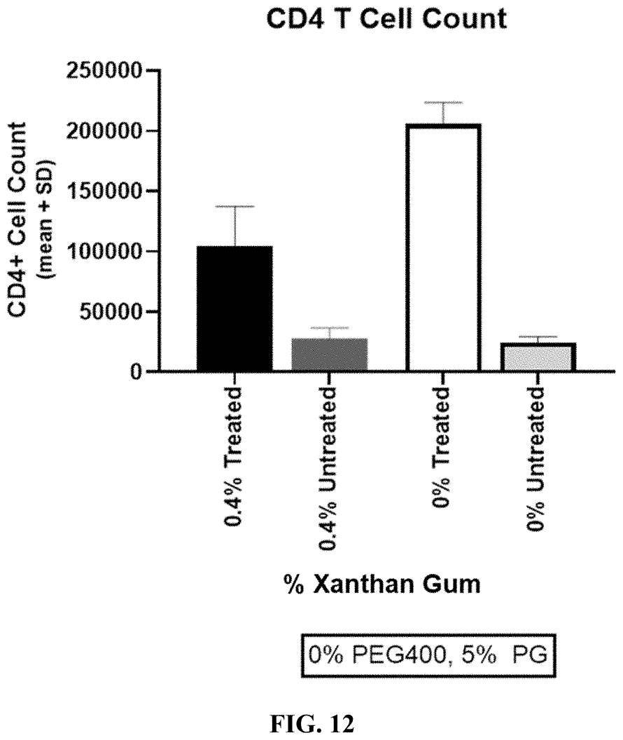

The percentage of Xanthan gum (XG) was investigated for a 0.1% Compound A formulation under conditions similar to Example 14. XG concentrations tested in the animal studies were 0% and 0.4%. The formulation without XG indicated an increase in inflammation compared to the formulation which contained 0.4% XG (FIG. 12). Not only did this demonstrate that XG reduces the inflammatory response of the formulation, but it was also discovered that the viscosity of lower XG concentration formulations were similar to a lotion instead of a cream upon scale up. The increased concentration of XG allowed for efficient scale up of the formulation.

TABLE 23

| | 0.4% XG | 0% XG |
|---|---|---|
| | % w/w | % w/w |
| Compound A HCl* | 0.0108 | 0.0108 |
| Purified Water, USP | qs | qs |
| Disodium EDTA, USP | 0.05 | 0.05 |
| PEG 400, NF | 0 | 0 |
| Propylene Glycol, USP | 15 | 15 |
| Xanthan Gum, NF | 0.4 | 0.0 |
| White Petrolatum, USP | 7 | 7 |
| Light Mineral Oil, NF | 4 | 4 |
| Glyceryl mono and distearate, NF | 3 | 3 |
| Cetyl Alcohol, NF | 3 | 3 |
| Stearyl Alcohol, NF | 1.75 | 1.75 |
| Med. Chain Triglycerides, NF | 6 | 6 |
| Polysorbate 20, NF | 1.25 | 1.25 |
| Phenoxyethanol, NF | 0.5 | 0.5 |
| Total | 100 | 100 |

### Example 22. Additional Formulations

TABLE 24
Representative PEG Ointment

| | | % w/w |
|---|---|---|
| Compound A | | 0.01-1% |
| PEG 400 | Solvent | 50.00 |
| PEG 3350 | Viscosity | 30.00 |
| Propylene Glycol | Solvent | 20.00 |

TABLE 25
Representative Film Forming Foam

| | | % w/w |
|---|---|---|
| Compound A | | 0.01-1% |
| Cocoyl Caprylocaprate | Emollient | 3.00 |
| Cetostearyl Alcohol | Foam stabilizer | 3.00 |
| Macrogol Cetostearyl Ether 20 | Foaming Agent | 5.00 |
| Polyvinylpyrrolidone | Film Former | 10.00 |
| Propane/Isobutane | Propellant | 6.00 |
| Deionized water | Solvent | 73.00 or q.s. |

TABLE 26
Representative Gel Formulation

| | | % w/w |
|---|---|---|
| Compound A | | 0.01-1% |
| Ethanol | Solvent | 10.00 |
| Propylene Glycol | Solvent | 10.00 |
| Isopropyl Myristate | Emollient | 2.00 |
| Glycerin | Solvent | 5.00 |
| Poloxamer 407 | Gelling agent | 15.00-20.00 |
| Deionized water | Solvent | 53.00-58.00 or q.s. |

TABLE 27
Representative Suppository

| | | % w/w |
|---|---|---|
| Compound A | | 0.01-1% |
| Gelatin | Gelling Agent | 14.00 |
| Glycerol | Solvent | 70.00 |
| Water | Solvent | 16.00 or q.s. |
| Sorbic acid | Preservative | 0.1% |

TABLE 28
Representative Lotion

| | | % w/w |
|---|---|---|
| Compound A | | 0.01-1% |
| Propylene glycol | Solvent | 5 |
| Xantham Gum | Thickener | 0.2 |
| Light Mineral Oil | Humectant | 4 |
| Glycerol monostearate | Emollient | 3 |
| Polysorbate 20 | Emulsifier | 1.25 |
| White petrolatum | Emmolient | 7 |
| Cetyl alcohol | Emulsifier | 3 |
| Stearyl Alcohol | Emulsifier | 1.75 |
| Medium chain triglycerides (MCT) | Solvent | 5 |
| EDTA | Chelating agent | 0.05 |
| Phenoxyethanol | Preservative | 0.5 |
| PEG400 | Solvent | 5 |
| Water | Solvent | 64 or q.s. |

TABLE 29
Representative Transdermal Patch

| | | % w/w |
|---|---|---|
| Compound A | | 0.01-1% |
| PEG300 | Solvent | 30.00 |
| Propylene glycol | Solvent | 15.00 |
| Water | Solvent | 10.00 or q.s. |
| Hydroxypropyl | Thickener | 22 |
| Methylcellulos | | |
| Polyvinylpyrrolidone | Thickener | 22 |

TABLE 30
Representative Hydrogel Formulation

| | % w/w |
|---|---|
| Compound A | 0.01-1% |
| PEG 400 | 5.0 |
| Glycol Chitosan | 3.0 |
| Water | 90.00 or q.s. |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A pharmaceutical composition, suitable for topical skin application to a human patient with a skin disorder, comprising:
(1) a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof; and
(2) a means for effecting skin permeation of the therapeutic agent or pharmaceutically acceptable salt thereof to the patient.

2. The pharmaceutical composition of claim 1, wherein the pharmaceutical composition is a cream, gel, hydrogel, aerosolized foam, non-aerosolized foam, film forming spray, or ointment.

3. The pharmaceutical composition of claim 1, wherein the pharmaceutical composition is a cream.

4. The pharmaceutical composition of claim 1, wherein the skin disorder is an immune mediated dermatological disease.

5. The pharmaceutical composition of claim 4, wherein the immune mediated dermatological disease is mycosis fungoides, atopic dermatitis, psoriasis, contact dermatitis, chronic hand eczema, hidradenitis suppurativa, lichen planus, acne, skin blistering disease, chronic urticaria, or cold induced urticaria.

6. The pharmaceutical composition of claim 4, wherein the immune mediated dermatological disease is atopic dermatitis.

7. The pharmaceutical composition of claim 4, wherein the immune mediated dermatological disease is psoriasis.

8. A pharmaceutical composition, comprising:
an oil-in-water emulsion; and
a therapeutically effective amount of a therapeutic agent which is (R)-4-(3-((S)-1-(4-amino-3-methyl-1H-pyrazolo[3, 4-d]pyrimidin-1-yl)ethyl)-5-chloro-2-ethoxy-6-fluorophenyl)pyrrolidin-2-one, or a pharmaceutically acceptable salt thereof.

9. The pharmaceutical composition of claim 8, wherein the oil-in-water emulsion comprises water, an oil component, and an emulsifier component.

10. The pharmaceutical composition of claim 9, wherein the oil component comprises about 10% to about 40% by weight of the composition.

11. The pharmaceutical composition of claim 9, wherein the oil component comprises one or more substances independently selected from petrolatums, fatty alcohols, mineral oils, triglycerides, and silicone oils.

12. The pharmaceutical composition of claim 9, wherein the oil component comprises one or more substances independently selected from white petrolatum, cetyl alcohol, stearyl alcohol, light mineral oil, and medium chain triglycerides.

13. The pharmaceutical composition of claim 9, wherein the oil component comprises an occlusive agent component.

14. The pharmaceutical composition of claim 13, wherein the occlusive agent component is present in an amount of about 1% to about 10% by weight of the composition.

15. The pharmaceutical composition of claim 13, wherein the occlusive agent component comprises a petrolatum.

16. The pharmaceutical composition of claim 13, wherein the occlusive agent component comprises white petrolatum.

17. The pharmaceutical composition of claim 9, wherein the oil component comprises a stiffening agent component.

18. The pharmaceutical composition of claim 17, wherein the stiffening agent component is present in an amount of about 1% to about 8% by weight of the composition.

19. The pharmaceutical composition of claim 17, wherein the stiffening agent component comprises one or more substances independently selected from fatty alcohols.

20. The pharmaceutical composition of claim 17, wherein the stiffening agent component comprises one or more substances independently selected from Cu-20 fatty alcohols.

21. The pharmaceutical composition of claim 17, wherein the stiffening agent component comprises one or more substances independently selected from cetyl alcohol and stearyl alcohol.

22. The pharmaceutical composition of claim 9, wherein the oil component comprises an emollient component.

23. The pharmaceutical composition of claim 22, wherein the emollient component is present in an amount of about 5% to about 15% by weight of the composition.

24. The pharmaceutical composition of claim 22, wherein the emollient component comprises one or more substances independently selected from mineral oils and triglycerides.

25. The pharmaceutical composition of claim 22, wherein the emollient component comprises one or more substances independently selected from light mineral oil and medium chain triglycerides.

26. The pharmaceutical composition of claim 8, wherein the water is present in an amount of about 30% to about 70% by weight of the composition.

27. The pharmaceutical composition of claim 9, wherein the emulsifier component is present in an amount of about 1% to about 10% by weight of the composition.

28. The pharmaceutical composition of claim 9, wherein the emulsifier component comprises one or more substances independently selected from glyceryl fatty esters and sorbitan fatty esters.

29. The pharmaceutical composition of claim 9, wherein the emulsifier component comprises one or more substances independently selected from glyceryl stearate and polysorbate 20.

30. The pharmaceutical composition of claim 9, wherein the emulsifier component comprises one or more substances independently selected from glyceryl monostearate, glyceryl distearate, and polysorbate 20.

31. The pharmaceutical composition of claim 8, wherein the pharmaceutical composition further comprises a stabilizing agent component.

32. The pharmaceutical composition of claim 31, wherein the stabilizing agent component is present in an amount of about 0.01% to about 2% by weight of the composition.

33. The pharmaceutical composition of claim 31, wherein the stabilizing agent component comprises one or more independently selected polysaccharides.

34. The pharmaceutical composition of claim 31, wherein the stabilizing agent component comprises xanthan gum.

35. The pharmaceutical composition of claim 8, wherein the pharmaceutical composition further comprises a solvent component.

36. The pharmaceutical composition of claim 35, wherein the solvent component is present in an amount of about 10% to about 30% by weight of the composition.

37. The pharmaceutical composition of claim 35, wherein the solvent component comprises one or more substances independently selected from alkylene glycols and polyalkylene glycols.

38. The pharmaceutical composition of claim 35, wherein the solvent component comprises one or more substances independently selected from propylene glycol and polyethylene glycol.

39. The pharmaceutical composition of claim 38, wherein the polyethylene glycol is PEG300.

40. The pharmaceutical composition of claim 8, wherein the therapeutic agent is present in an amount of about 0.001% to about 1.0% by weight of the composition on a free base basis.

41. The pharmaceutical composition of claim 8, comprising:
about 30% to about 70% of water by weight of the composition;
about 10% to about 40% of an oil component by weight of the composition;
about 1% to about 10% of an emulsifier component by weight of the composition;
about 10% to about 30% of a solvent component by weight of the composition;
about 0.01% to about 2% of a stabilizing agent component by weight of the composition; and
about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

42. The pharmaceutical composition of claim 41, wherein:
the oil component comprises one or more substances independently selected from petrolatums, fatty alcohols, mineral oils, triglycerides, and silicone oils;
the emulsifier component comprises one or more substances independently selected from glyceryl fatty esters and sorbitan fatty esters;
the solvent component comprises one or more substances independently selected from alkylene glycols and polyalkylene glycols; and
the stabilizing agent component comprises one or more independently selected polysaccharides.

43. The pharmaceutical composition of claim 41, wherein:
the oil component comprises one or more substances independently selected from white petrolatum, cetyl alcohol, stearyl alcohol, light mineral oil, and medium chain triglycerides;
the emulsifier component comprises one or more substances independently selected from glyceryl stearate and polysorbate 20;
the solvent component comprises one or more substances independently selected from propylene glycol and polyethylene glycol; and
the stabilizing agent component comprises xanthan gum.

44. The pharmaceutical composition of claim 8, comprising:
about 30% to about 70% of water by weight of the composition;
about 1% to about 10% of an occlusive agent component by weight of the composition;
about 1% to about 8% of a stiffening agent component by weight of the composition;
about 5% to about 15% of an emollient component by weight of the composition;
about 1% to about 10% of an emulsifier component by weight of the composition;
about 0.01% to about 2% of a stabilizing agent component by weight of the composition;
about 10% to about 30% of a solvent component by weight of the composition; and
about 0.001% to about 1.0% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt, by weight of the composition on a free base basis.

45. The pharmaceutical composition of claim 44, wherein:
the occlusive agent component comprises a petrolatum;
the stiffening agent component comprises one or more substances independently selected from one or more fatty alcohols;
the emollient component comprises one or more substances independently selected from mineral oils and triglycerides;
the emulsifier component comprises one or more substances independently selected from glyceryl fatty esters and sorbitan fatty esters;
the stabilizing agent component comprises one or more substances independently selected from polysaccharides; and
the solvent component comprises one or more substances independently selected from alkylene glycols and polyalkylene glycols.

46. The pharmaceutical composition of claim 44, wherein:
the occlusive agent component comprises white petrolatum;
the stiffening agent component comprises one or more substances independently selected from cetyl alcohol and stearyl alcohol;
the emollient component comprises one or more substances independently selected from light mineral oil and medium chain triglycerides;
the emulsifier component comprises one or more substances independently selected from glyceryl stearate and polysorbate 20;
the stabilizing agent component comprises xanthan gum; and
the solvent component comprises one or more substances independently selected from propylene glycol and polyethylene glycol.

47. The pharmaceutical composition of claim 44, wherein:
the occlusive agent component comprises white petrolatum;
the stiffening agent component comprises cetyl alcohol and stearyl alcohol;
the emollient component comprises light mineral oil and medium chain triglycerides;
the emulsifier component comprises glyceryl stearate and polysorbate 20;
the stabilizing agent component comprises xanthan gum; and
the solvent component comprises propylene glycol and polyethylene glycol.

48. The pharmaceutical composition of claim 8, wherein the therapeutic agent is present in an amount of about 0.001% by weight of the composition on a free base basis.

49. The pharmaceutical composition of claim 8, wherein the therapeutic agent is present in an amount of about 0.01% by weight of the composition on a free base basis.

50. The pharmaceutical composition of claim 8, wherein the therapeutic agent is present in an amount of about 0.1% by weight of the composition on a free base basis.

51. The pharmaceutical composition of claim 8, wherein the therapeutic agent is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt.

52. The pharmaceutical composition of claim 8, further comprising an antimicrobial preservative component.

53. The pharmaceutical composition of claim 52, wherein the antimicrobial preservative component is present in an amount of about 0.05% to about 2% by weight of the composition.

54. The pharmaceutical composition of claim 52, wherein the antimicrobial preservative component comprises phenoxyethanol.

55. The pharmaceutical composition of claim 8, further comprising a chelating agent component.

56. The pharmaceutical composition of claim 55, wherein the chelating agent component is present in an amount of about 0.01% to about 0.1% by weight of the composition.

57. The pharmaceutical composition of claim 55, wherein the chelating agent component comprises disodium EDTA.

58. The pharmaceutical composition of claim 8, comprising:
about 56% of water by weight of the composition;
about 7% of white petrolatum by weight of the composition;
about 3% of cetyl alcohol by weight of the composition;
about 1.75% of stearyl alcohol by weight of the composition;
about 4% of light mineral oil by weight of the composition;
about 6% of medium chain triglycerides by weight of the composition;
about 3% of glyceryl mono and distearate by weight of the composition;
about 1.25% of polysorbate 20 by weight of the composition;
about 0.35% of xanthan gum by weight of the composition;
about 7% of PEG300 by weight of the composition;
about 10% of propylene glycol by weight of the composition; and
about 0.01% by weight of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt, by weight of the composition.

59. The pharmaceutical composition of claim 8, wherein the composition is a cream, gel, hydrogel, aerosolized foam, non-aerosolized foam, film forming spray, or ointment.

60. The pharmaceutical composition of claim 8, wherein the composition is a cream.

61. A method of treating a skin disorder in a patient in need thereof, comprising applying a pharmaceutical composition of claim 8, to an area of skin of the patient.

62. A method of treating a skin disorder in a human patient in need thereof, comprising applying to the patient's skin a pharmaceutically acceptable composition comprising a therapeutically effective amount of a therapeutic agent which is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, or a pharmaceutically acceptable salt thereof, and a means of effecting skin permeation of the therapeutic agent to the patient, wherein the treating step is one or more of (i) inhibiting the skin disorder, and (b) ameliorating the skin disorder.

63. The method of claim 61, wherein the skin disorder is an immune mediated dermatological disease.

64. The method of claim 63, wherein the immune mediated dermatological disease is mycosis fungoides, atopic dermatitis, psoriasis, contact dermatitis, chronic hand eczema, hidradenitis suppurativa, lichen planus, acne, skin blistering disease, chronic urticaria, or cold induced urticaria.

65. The method of claim 63, wherein the immune mediated dermatological disease is atopic dermatitis.

66. The method of claim 63, wherein the immune mediated dermatological disease is psoriasis.
